(19) United States
(12) Patent Application Publication
(10) Pub. No.: US 2023/0020120 A1
(43) Pub. Date: Jan. 19, 2023

(54) MEASUREMENT DEVICE AND MEASUREMENT SYSTEM

(71) Applicant: Murata Manufacturing Co., Ltd., Nagaokakyo-shi (JP)

(72) Inventors: Jun TAKAGI, Nagaokakyo-shi (JP); Kiyoshi KURIHARA, Nagaokakyo-shi (JP); Tomoki TAKAHASHI, Nagaokakyo-shi (JP)

(21) Appl. No.: 17/944,564

(22) Filed: Sep. 14, 2022

Related U.S. Application Data
(63) Continuation of application No. PCT/JP2020/037817, filed on Oct. 6, 2020.

(30) Foreign Application Priority Data
Mar. 19, 2020 (JP) 2020-049738

(51) Int. Cl. A61B 5/252; A61B 5/00

(52) U.S. Cl. CPC A61B 5/252; A61B 5/682; A61B 2562/029

(57) ABSTRACT

A measurement device is provided that has a contact surface that comes into contact with a measurement portion of a living body. The measurement device includes a biosensor disposed on the contact surface and has a detection surface that acquires biological information. Moreover, a suction portion is provided that sucks the living body from one or multiple suction holes provided in the contact surface on a periphery of the detection surface of the biosensor.

### CROSS REFERENCE TO RELATED APPLICATIONS

This application is a continuation of PCT Application No. PCT/JP2020/037817, filed Oct. 6, 2020, which claims priority to Japanese Patent Application No. 2020-049738, filed Mar. 19, 2020, the entire contents of each of which are hereby incorporated by reference in their entirety.

### TECHNICAL FIELD

The present invention relates to a measurement device and a measurement system.

### BACKGROUND

International Publication No. 2015/125222 (hereinafter “Patent Document 1”) discloses an intraoral moisture measurement instrument. The intraoral moisture measurement instrument described therein includes a swing member, a moisture amount detection unit provided at a distal end of the swing member, and a biasing member to bias the swing member in one swing direction.

However, in recent years, there has been a demand and need for a measurement device and a measurement system with increased measurement accuracy.

### SUMMARY OF THE INVENTION

Accordingly, a measurement device of an exemplary aspect of the present invention has a contact surface that comes into contact with a measurement portion of a living body, and includes a biosensor disposed on the contact surface that has a detection surface that acquires biological information and a suction portion that sucks the living body from one or multiple suction holes provided in the contact surface on a periphery of the detection surface of the biosensor.

In another exemplary aspect, a measurement system is provided that includes a measurement device having a contact surface that come into contact with a measurement portion of a living body and a processing device that communicates with the measurement device. In this aspect, the measurement device includes a biosensor disposed on the contact surface and has a detection surface that acquires biological information, a suction portion that sucks the living body from one or multiple suction holes provided in the contact surface on a periphery of the detection surface of the biosensor, and a first communication unit that transmits the biological information to the processing device. The processing device includes a second communication unit that receives the biological information from the first communication unit of the measurement device and a calculation unit that calculates an amount of a measurement target on the basis of the biological information.

According to the exemplary aspects of the present invention, a measurement device and a measurement system are provided with increased measurement accuracy.

### BRIEF DESCRIPTION OF DRAWINGS

FIG. 1 is a schematic perspective view of a measurement device of Embodiment 1 according to an exemplary aspect.

FIG. 2 is a schematic diagram of an internal configuration of an example of the measurement device of Embodiment 1 according to an exemplary aspect.

FIG. 3 is a diagram of a schematic configuration of an example of the measurement device of Embodiment 1 according to an exemplary aspect.

FIG. 4 is a block diagram of a schematic configuration of an example of the measurement device of Embodiment 1 according to an exemplary aspect.

FIG. 5 is a flowchart of an example of an operation of the measurement device of Embodiment 1 according to an exemplary aspect.

FIG. 6 is a schematic view of an example of a state in which the measurement device of Embodiment 1 can be used according to an exemplary aspect.

FIG. 7 is a schematic diagram of an example of a cover film.

FIG. 8A is a schematic diagram of an example of a state in which the measurement device of Embodiment 1 is brought into contact with a living body.

FIG. 8B is a schematic diagram of an example of a state in which the measurement device of Embodiment 1 is brought into contact with a living body.

FIG. 9 is a schematic enlarged diagram of part of a measurement device of a modification of Embodiment 1 according to an exemplary aspect.

FIG. 10 is a schematic enlarged diagram of part of a measurement device of a modification of Embodiment 1 according to an exemplary aspect.

FIG. 11 is a schematic enlarged diagram of part of a measurement device of a modification of Embodiment 1 according to an exemplary aspect.

FIG. 12 is a schematic enlarged diagram of part of a measurement device of a modification of Embodiment 1 according to an exemplary aspect.

FIG. 13 is a schematic enlarged diagram of part of a measurement device of Embodiment 2 according to an exemplary aspect.

FIG. 14 is a schematic enlarged diagram of part of a measurement device of a modification of Embodiment 2 according to an exemplary aspect.

FIG. 15 is a schematic enlarged diagram of part of a measurement device of Embodiment 3 according to an exemplary aspect.

FIG. 16 is a schematic enlarged diagram of part of a measurement device of a modification of Embodiment 3 according to an exemplary aspect.

FIG. 17 is a schematic enlarged diagram of part of a measurement device of Embodiment 4 according to an exemplary aspect.

FIG. 18 is a schematic enlarged diagram of part of a measurement device of Embodiment 5 according to an exemplary aspect.

FIG. 19 is a schematic enlarged diagram of part of a measurement device of Embodiment 6 according to an exemplary aspect.

FIG. 20 is a schematic enlarged diagram of part of a measurement device of a modification of Embodiment 6 according to an exemplary aspect.

FIG. 21 is a schematic diagram of an internal configuration of an example of a measurement device of Embodiment 7 according to an exemplary aspect.

FIG. 22 is a block diagram of a schematic configuration of an example of the measurement device of Embodiment 7 according to an exemplary aspect.

FIG. 22A is a block diagram of a schematic configuration of a measurement device of a modification of Embodiment 7 according to an exemplary aspect.

FIG. 23 is a flowchart of an example of an operation of the measurement device of Embodiment 7 according to an exemplary aspect.

FIG. 24 is a schematic diagram of an internal configuration of an example of a measurement device of Embodiment 8 according to an exemplary aspect.

FIG. 25 is a block diagram of a schematic configuration of an example of the measurement device of Embodiment 8 according to an exemplary aspect.

FIG. 26 is a graph of an example of a relationship between suction pressure and measurement value variation.

FIG. 27 is a flowchart of an example of an operation of the measurement device of Embodiment 8 according to an exemplary aspect.

FIG. 28 is a schematic diagram of an internal configuration of an example of a measurement device of Embodiment 9 according to an exemplary aspect.

FIG. 29 is a block diagram of a schematic configuration of an example of the measurement device of Embodiment 9 according to an exemplary aspect.

FIG. 30 is a flowchart of an example of an operation of the measurement device of Embodiment 9 according to an exemplary aspect.

FIG. 31 is a schematic diagram of an internal configuration of an example of a measurement device of Embodiment 10 according to an exemplary aspect.

FIG. 32 is a schematic enlarged diagram of part of the measurement device of Embodiment 10 according to an exemplary aspect.

FIG. 33 is a schematic diagram of an example of a state in which the measurement device of Embodiment 10 according to an exemplary aspect.

FIG. 34 is a schematic diagram of an internal configuration of an example of a measurement device of Embodiment 11 according to an exemplary aspect.

FIG. 35 is a schematic diagram of an internal configuration of a measurement device of a modification of Embodiment 11 according to an exemplary aspect.

FIG. 36 is a schematic diagram of an internal configuration of an example of a measurement device of Embodiment 12 according to an exemplary aspect.

FIG. 37 is a block diagram of a schematic configuration of an example of a measurement system of Embodiment 13 according to an exemplary aspect.

FIG. 38 is a flowchart of an example of an operation of the measurement system of Embodiment 13 according to an exemplary aspect.

### DETAILED DESCRIPTION OF EMBODIMENTS

As described above, an intraoral moisture measurement instrument described in Patent Document 1 is known as an example of a measurement device. This intraoral moisture measurement instrument measures moisture in an oral cavity by bringing the intraoral moisture measurement instrument into direct or indirect contact with the measurement portion in the oral cavity.

However, as in the device described in Patent Document 1, there is an issue in that when the contact between the detection surface of a sensor and the measurement portion is insufficient, the accuracy of the measurement of moisture amount decreases. For example, the contact angle between the detection surface of the sensor and a living body varies depending on such as user usage. Thus, the degree of contact changes for each measurement, and the measurement value may vary as a result. Further, depending on the measurement portion, it may be difficult to maintain the state in which the detection surface of the sensor is in contact with a living body.

Further, when the detection surface of the sensor is brought into contact with the measurement portion in an oral cavity of a living body, there is a problem in that it is hard for a user to visually confirm the placement and use. Moreover, even when it is possible to visually confirm, because a living body as the measurement target has unevenness, there is a problem in that it is hard to objectively determine whether or not the contact is accurate, that is, whether or not the contact is made to an extent that the measurement accuracy may be ensured.

In view of the foregoing, exemplary configurations including a suction portion that suctions a living body are provided according to exemplary embodiments.

Thus, a measurement device is provided in one exemplary aspect that has a contact surface that comes into contact with a measurement portion of a living body, includes a biosensor disposed on the contact surface and has a detection surface that acquires biological information and a suction portion that sucks the living body from one or multiple suction holes provided in the contact surface on a periphery of the detection surface of the biosensor. With the configuration above, it is possible to suck a living body by the suction portion and bring the living body into contact with the detection surface of the biosensor. As a result, the measurement accuracy of the device is increased.

The measurement device may further include a housing having a longer direction, in which the housing may include a sensor portion provided on one end side in the longer direction and a grip portion provided on another end side in the longer direction. Moreover, the biosensor may be disposed in the sensor portion, and the multiple suction holes may be provided to sandwich the biosensor in the longer direction. With the configuration above, a living body can easily be brought into contact with the detection surface of the biosensor in the longer direction of the housing. As such, the measurement accuracy can further be increased.

The measurement device may further include a housing having a longer direction, in which the housing may include a sensor portion provided on one end side in the longer direction and a grip portion provided on another end side in the longer direction. Moreover, the biosensor may be disposed in the sensor portion, and the multiple suction holes may be provided to sandwich the biosensor in a shorter direction orthogonal to the longer direction. With the configuration above, a living body may easily be brought into contact with the detection surface of the biosensor in the shorter direction of the housing. With this configuration, the measurement accuracy can further be increased.

The suction portion may suck the living body from one or multiple sensor suction holes provided in the detection surface of the biosensor.

In addition, the detection surface of the biosensor may have a polygonal shape, and the multiple suction holes may be provided in corner portions of the detection surface. With the configuration above, a living body may more easily be brought into contact with the detection surface of the biosensor. With this configuration, the measurement accuracy can further be increased.

The multiple suction holes may symmetrically be provided with respect to the biosensor. With the configuration above, a living body may more easily be brought into contact with the detection surface of the biosensor and the measurement accuracy can further be increased.

The suction portion may include a pump that sucks gas, a suction path connecting the one or multiple suction holes and the pump, and one or multiple filters disposed in the one or multiple suction holes and/or the suction path and isolating liquid and gas from each other. With the configuration above, the inflow of liquid into the measurement device may be suppressed.

The one or multiple filters may be hydrophobic air-permeable membranes. With the configuration above, the inflow of liquid into the measurement device may further be suppressed.

Yet further, the measurement device may include a step or step portion protruding from the contact surface toward outside the measurement device and being provided on a periphery of the biosensor and the one or multiple suction holes. With the configuration above, a living body may more easily be brought into contact with the detection surface of the biosensor and the measurement accuracy can further be increased.

The measurement device may further include a calculation unit that calculates an amount of a measurement target on the basis of the biological information acquired by the biosensor. With the configuration above, the amount of the measurement target can be calculated.

Moreover, the amount of the measurement target may be a moisture amount.

With the configuration above, the moisture amount can be measured.

The measurement device may further include a pressure detection unit that detects a suction pressure with which the suction portion sucks the living body and a processing unit that outputs trigger information for starting measurement on the basis of the suction pressure detected by the pressure detection unit. With the configuration above, the measurement can be started on the basis of the suction pressure and the measurement accuracy can further be increased.

The processing unit may output the trigger information for starting measurement when the suction pressure is 10 kPa or more and 40 kPa or less. With the configuration above, the measurement variation can be suppressed and the measurement accuracy can further be increased.

The biosensor may be an electrostatic capacity sensor that detects electrostatic capacity, and the processing unit may convert electrostatic capacity detected by the electrostatic capacity sensor into frequency. With the configuration above, the measurement accuracy can be increased.

The measurement device may further include a contact detection unit that detects contact information between the biosensor and the living body, in which the suction portion may start sucking on the basis of contact information detected by the contact detection unit. With the configuration above, the suction can be started after the contact is detected.

Moreover, the measurement device may further include a cover film that covers the biosensor and the one or multiple suction holes, in which the cover film may have a membrane portion that isolates liquid and gas from each other. With the configuration above, the inflow of liquid into the measurement device can be suppressed.

The measurement portion of the living body may be a measurement portion in the oral cavity. With the configuration above, the inside of the oral cavity can be measured.

In another exemplary aspect, a measurement system is provided that includes a measurement device having a contact surface that comes into contact with a measurement portion of a living body and a processing device that communicates with the measurement device, in which the measurement device includes a biosensor disposed on the contact surface and having a detection surface that acquires biological information, a suction portion that sucks the living body from one or multiple suction holes provided in the contact surface on a periphery of the detection surface of the biosensor, and a first communication unit that transmits the biological information to the processing device. In this exemplary aspect, the processing device includes a second communication unit that receives the biological information from the first communication unit of the measurement device and a calculation unit that calculates an amount of a measurement target on the basis of the biological information. With the configuration above, a living body can be sucked by the suction portion and the living body can be brought into contact with the detection surface of the biosensor. With this configuration, the measurement accuracy can be increased.

Hereinafter, exemplary embodiments of the present invention will be described with reference to the accompanying drawings. Note that the following description is merely exemplary in nature and is not intended to limit the present disclosure, applications thereof, or uses thereof. Further, the drawings are schematic, and such as a ratio of respective sizes does not necessarily match the actual one.

### Embodiment 1

[Overall Configuration]

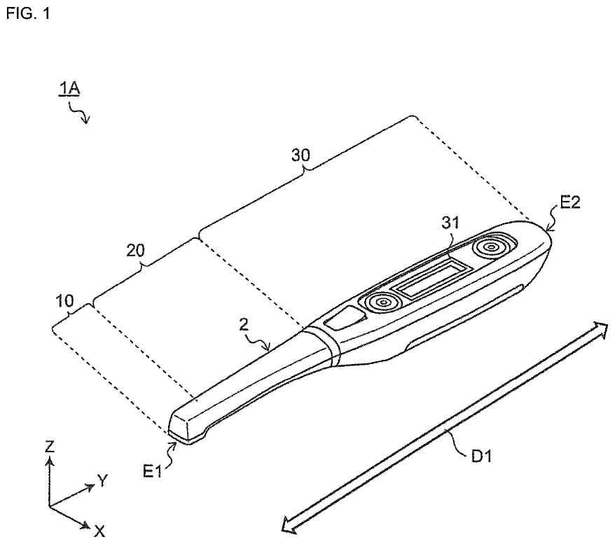

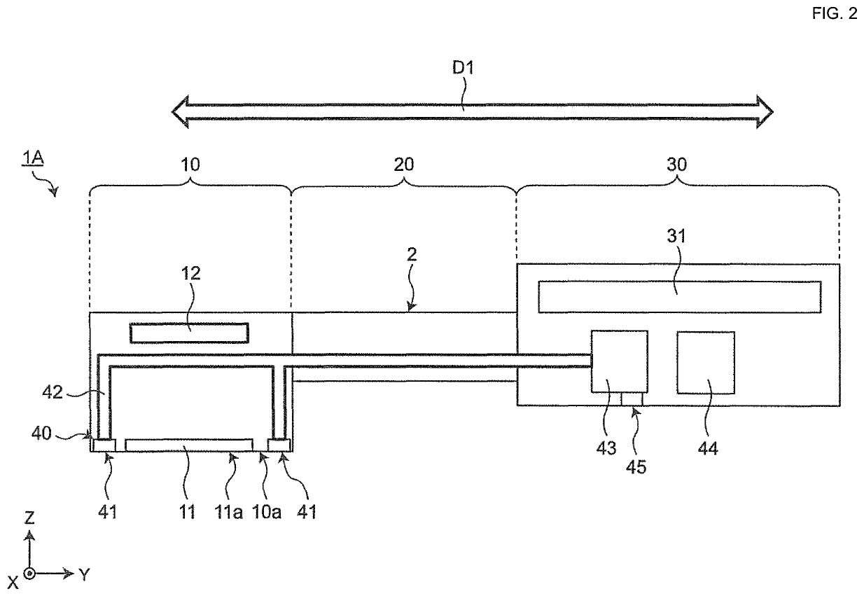

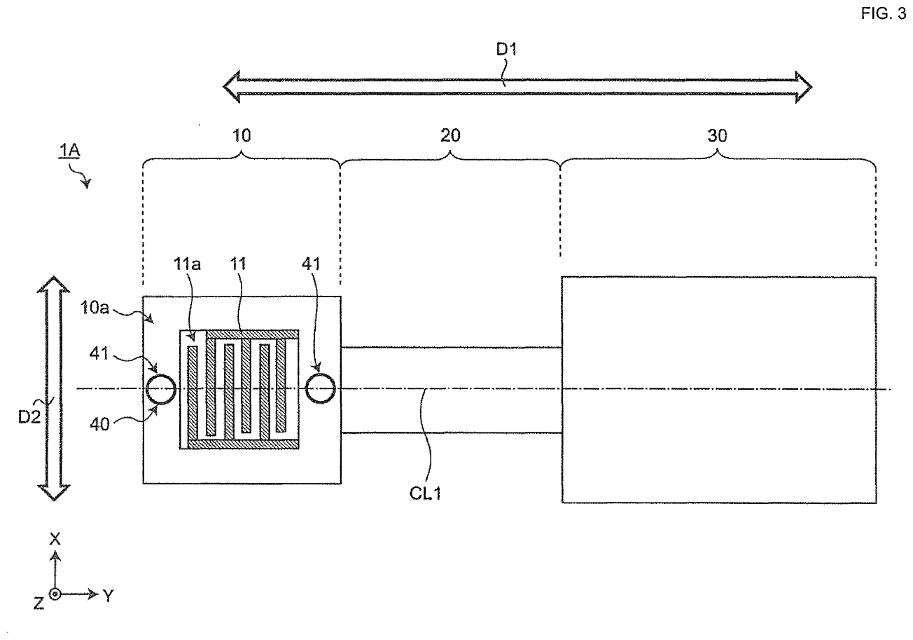

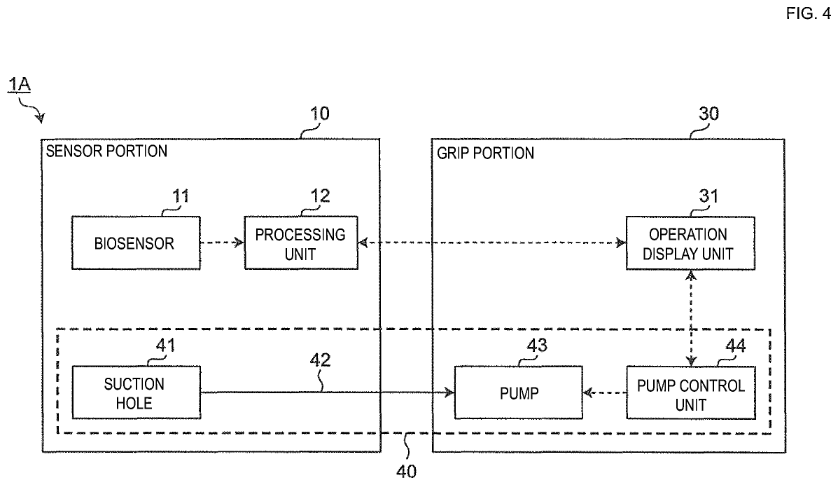

FIG. 1 is a schematic perspective view of a measurement device 1A of Embodiment 1 according to an exemplary aspect. FIG. 2 is a schematic diagram of an internal configuration of an example of the measurement device 1A of Embodiment 1. FIG. 3 is a diagram of a schematic configuration of an example of the measurement device 1A of Embodiment 1. FIG. 4 is a block diagram of a schematic configuration of an example of the measurement device 1A of Embodiment 1. In general, X, Y, and Z directions in the drawings respectively indicate a width direction, a length direction, and a height direction of the measurement device 1A. Further, a D1 direction in the drawings indicates a longer direction of the measurement device 1A, and a D2 direction indicates a shorter direction of the measurement device 1A.

In Embodiment 1, an example will be described in which the measurement device 1A is an intraoral measurement device. Further, in Embodiment 1, an example will be described in which the measurement target of the measurement device 1A is moisture and the moisture amount in an oral cavity is measured using the measurement device 1A.

<Appearance>

The appearance of the measurement device 1A will be described. As illustrated in FIG. 1 to FIG. 3, the measurement device 1A includes a housing 2. The housing 2 has a rod shape having a longer direction D1. Specifically, the housing 2 includes a sensor portion 10 (also referred to as a “sensor”), a probe portion 20 (also referred to as a “probe”), and a grip portion 30 (also referred to as a “grip”).

The sensor portion 10 is a portion that comes into contact with the measurement portion of a living body. The measurement portion of a living body is a measurement portion in an oral cavity, for example, and can be a tongue portion, for example. The sensor portion 10 is provided at one end E1 in the longer direction D1 of the measurement device 1A. Outer sizes of the sensor portion 10 are designed to be smaller than those of the probe portion 20 and the grip portion 30. For example, the sizes of the sensor portion 10 in the X direction and the Y direction are designed to be smaller than those of the probe portion 20 and the grip portion 30.

The sensor portion 10 has a contact surface 10a that comes into contact with a measurement portion of a living body. The contact surface 10a is provided on one end E1 side in the longer direction D1 of the housing 2 and is provided in directions (X and Y directions) intersecting with an end surface on the one end E1 side.

The probe portion 20 connects the sensor portion 10 and the grip portion 30. The probe portion 20 is formed in a rod shape in the exemplary aspect. The size of the X direction and the size of the Z direction of the probe portion 20 decrease from the grip portion 30 toward the sensor portion 10. That is, the probe portion 20 has a shape becoming gradually narrower from the grip portion 30 toward the sensor portion 10.

The grip portion 30 is a portion gripped by a user. The grip portion 30 is provided at the other end E2 of the measurement device 1A in the longer direction D1. The grip portion 30 is formed in a rod shape. Outer sizes of the grip portion 30 are designed to be larger than those of the sensor portion 10 and the probe portion 20. For example, the sizes of the grip portion 30 in the X, Y, and Z directions are designed to be larger than those of the sensor portion 10 and the probe portion 20.

The housing 2 is made of resin, for example. Further, part or an entirety of the housing 2 may be formed of metal in alternative exemplary aspects.

Next, components forming the measurement device 1A will be described. As illustrated in FIG. 1 to FIG. 4, the measurement device 1A includes a biosensor 11, a processing unit 12, an operation display unit 31, and a suction portion 40.

Note that, in Embodiment 1, an example will be described in which the measurement device 1A includes the operation display unit 31, but the present invention is not limited thereto. The operation display unit 31 is not an essential components and may be included in a device different from the measurement device 1A.

<Biosensor>

The biosensor 11 acquires biological information. The biological information is various physiological and anatomical information that a living body generates, for example. In particular, the biological information is information such as electrostatic capacity, a resistance value, moisture amount, temperature, hardness, sound, and light, for example. The biosensor 11 comes into contact with a measurement portion of a living body and acquires biological information of the measurement portion where the biosensor 11 comes into contact.

In Embodiment 1, the biosensor 11 is an electrostatic capacity sensor, for example. The biosensor 11 comes into contact with the measurement portion in the oral cavity and acquires information on electrostatic capacity. That is, in Embodiment 1, the biological information acquired by the biosensor 11 is information on electrostatic capacity.

The biosensor 11 is disposed on the contact surface 10a on the one end E1 side in the longer direction D1 of the measurement device 1A. For example, the biosensor 11 is disposed to a recessed portion provided on a side of the contact surface 10a of the sensor portion 10 of the housing 2.

Moreover, the biosensor 11 is formed in a planar shape to have a detection surface 11a that acquires biological information. The detection surface 11a is exposed on the side of the contact surface 10a of the sensor portion 10. A comb-shaped electrode is disposed on the detection surface 11a in an exemplary aspect.

For example, the detection surface 11a is formed in a rectangular shape when viewed from the height direction (i.e., the Z direction) of the measurement device 1A. The detection surface 11a detects biological information by coming into contact with a measurement portion. That is, the biosensor 11 acquires biological information by bringing the detection surface 11a into contact with a measurement portion. Then, the biological information acquired by the biosensor 11 is transmitted to the processing unit 12.

<Processing Unit>

The processing unit 12 converts the biological information acquired by the biosensor 11, and outputs the converted information.

The processing unit 12 converts analog information acquired by the biosensor 11 into digital information. In Embodiment 1, the processing unit 12 includes a frequency conversion circuit to convert information on electrostatic capacity acquired by the biosensor 11 into frequency. For example, the processing unit 12 repeatedly charges and discharges the biosensor 11 regarded as electrostatic capacity, and the information on electrostatic capacity is converted into the frequency of the cycle determined by the charging and discharging speed. Thus, the processing unit 12 outputs a value on frequency as an output value of the biosensor 11.

The processing unit 12 transmits the converted information to a calculation unit. The calculation unit calculates the amount of the measurement target on the basis of the converted information. The calculation unit may be included in the measurement device 1A or may be included in a device different from the measurement device 1A.

In an exemplary aspect, the processing unit 12 may be implemented with such as a semiconductor element. Moreover, the processing unit 12 can comprise a microcomputer, a CPU, an MPU, a GPU, a DSP, an FPGA, an ASIC, a discrete semiconductor, or an LSI, for example. The function of the processing unit 12 may be formed only by hardware or may be implemented with a combination of hardware and software. The processing unit 12 achieves a predetermined function by reading out data or programs stored in a storage unit (not illustrated) in the processing unit 12 and performing various arithmetic processes. The storage unit may be implemented with a hard disk (HDD), an SSD, a RAM, a DRAM, a ferroelectric memory, a flash memory, a magnetic disk, or a combination thereof, for example.

The processing unit 12 converts the biological information acquired by the biosensor 11, and stores the converted information in the storage unit. The processing unit 12 transmits the information stored in the storage unit to the calculation unit. For example, the processing unit 12 transmits information to the calculation unit on the basis of trigger information for starting the measurement. The trigger information for starting the measurement may be generated on the basis of contact information between the biosensor 11 and the measurement portion of a living body, the suction pressure of the suction portion 40, and/or the input information inputted to the operation display unit 31, for example. Moreover, the processing unit 12 can be disposed inside the sensor portion 10 in an exemplary aspect.

<Operation Display Unit>

The operation display unit 31 receives an input from a user and displays information on the amount of the measurement target. For example, the operation display unit 31 includes an operation unit that receives an operation from a user and a display unit to display information.

The operation unit includes one or multiple buttons that receives an input from a user. The multiple buttons include buttons such as a power button to switch power ON/OFF, a suction start button to start sucking by the suction portion 40, a suction stop button to stop sucking by the suction portion 40, and/or a measurement start button to start measurement, for example.

The display unit displays information on the amount of the measurement target and can be a display, for example. The information on the amount of the measurement target is transmitted from the calculation unit to the display unit included in the measurement device 1A, for example. Alternatively, the information on the amount of the measurement target is transmitted from a calculation unit included in a device different from the measurement device 1A to the display unit, for example, via such as a network. In an exemplary aspect, the operation display unit 31 is disposed on an upper surface of the grip portion 30.

<Suction Portion>

The suction portion 40 is configured to suck a living body. The suction portion 40 sucks a living body from multiple suction holes 41 provided on a periphery of the detection surface 11a of the biosensor 11 on the contact surface 10a. In Embodiment 1, the two suction holes 41 are provided in the contact surface 10a.

As illustrated in FIG. 2 and FIG. 3, the multiple suction holes 41 are provided along the longer direction D1 (i.e., the Y direction) of the housing 2. Specifically, the multiple suction holes 41 are provided to sandwich the biosensor 11 in the longer direction D1 of the housing 2. That is, in the longer direction D1 of the housing 2, the multiple suction holes 41 are provided on both sides of the biosensor 11. The multiple suction holes 41 and the biosensor 11 are provided in the order of the suction hole 41, the biosensor 11, and the suction hole 41 from the one end E1 side toward the other end E2 in the longer direction D1 of the housing 2.

In Embodiment 1, the multiple suction holes 41 are provided along an axial line CL1 in the longer direction D1 of the housing 2 when viewed from the height direction (i.e., Z direction) of the measurement device 1A. In the exemplary aspect, the axial line CL1 is a line that extends in the longer direction D1 of the housing 2 and passes through a center of the measurement device 1A when the measurement device 1A is viewed from the side of the contact surface 10a.

As further shown, the multiple suction holes 41 are symmetrically provided with respect to the biosensor 11. Specifically, the multiple suction holes 41 are symmetrically provided with respect to the biosensor 11 in the longer direction D1 of the housing 2. For example, the multiple suction holes 41 are formed in a circular shape. Further, the sizes of the multiple suction holes 41 are the same.

In addition, the suction portion 40 includes a suction path 42, a pump 43, and a pump control unit 44.

The suction path 42 is a path that connects the multiple suction holes 41 and the pump 43. The suction path 42 is formed of a hollow tubular member, such as a tube or a pipe. The suction path 42 has multiple inlet paths connected to the multiple suction holes 41 and an outlet path connected to the multiple inlet paths and the pump 43. That is, the multiple inlet paths merge into the outlet path.

The suction path 42 is disposed in the housing 2 across the sensor portion 10, the probe portion 20, and the grip portion 30.

The pump 43 is configured to suck gas during operation. The pump 43 sucks gas from the multiple suction holes 41 via the suction path 42. For example, the pump 43 is a piezoelectric pump, which advantageously provides that a minute pressure can easily be controlled.

The pump 43 is disposed inside the grip portion 30. Further, the grip portion 30 is provided with an exhaust hole 45 to discharge the gas sucked by the pump 43.

The pump control unit 44 controls the pump 43. For example, the pump control unit 44 controls the start and stop of sucking, and a suction pressure P1 of the pump 43. The pump control unit 44 may be implemented with such as a semiconductor element. For example, the pump control unit 44 may be formed of a microcomputer.

In Embodiment 1, the pump control unit 44 controls the pump 43 on the basis of the operation of the operation display unit 31. For example, input information such as suction start, suction stop, and setting of the suction pressure P1 are inputted to the operation display unit 31. The pump control unit 44 controls the pump 43 on the basis of the input information inputted to the operation display unit 31.

The measurement device 1A includes a control unit to integrally control the components constituting the measurement device 1A. The control unit includes a memory storing a program and a processing circuit corresponding to a processor such as a central processing unit (CPU), for example. For example, in the control unit, the processor executes the program stored in the memory. In Embodiment 1, the control unit controls the biosensor 11, the processing unit 12, the operation display unit 31, and the pump control unit 44.

[Operation of Measurement Device]

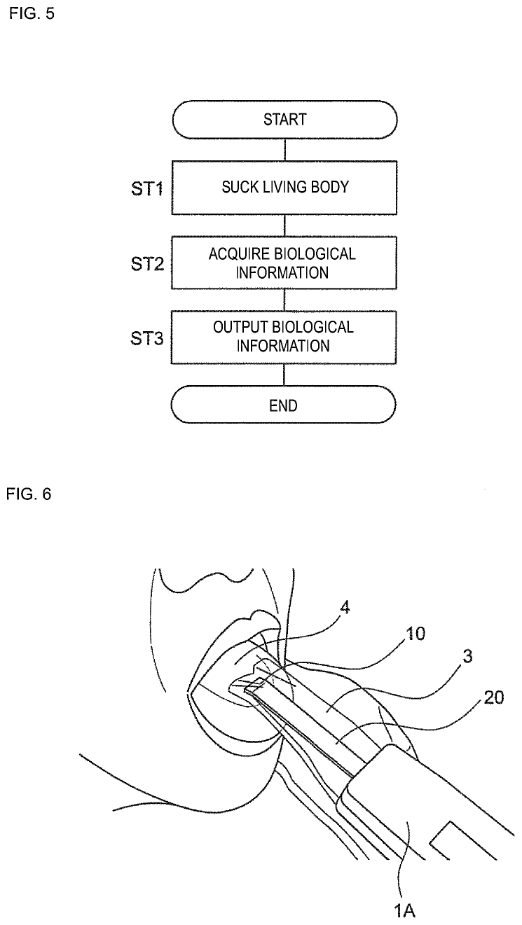

An example of an operation of the measurement device 1A, that is, an example of a measurement method will be described. FIG. 5 is a flowchart of an example of an operation of the measurement device 1A of Embodiment 1 according to the present invention.

As illustrated in FIG. 5, in step ST1, the suction portion 40 sucks a living body. In step ST1, the input information for starting suction is inputted to the operation display unit 31, for example. The pump control unit 44 controls the pump 43 on the basis of the input information for starting suction, and starts sucking. The pump 43 sucks gas from the multiple suction holes 41 via the suction path 42. With this, when the contact surface 10a of the sensor portion 10 of the measurement device 1A is in contact with a living body or is disposed in the vicinity thereof, the living body is sucked by the multiple suction holes 41.

In step ST2, the biosensor 11 acquires biological information. The biological information acquired by the biosensor 11 is transmitted to the processing unit 12.

For example, step ST2 is started when the power is turned on with the operation display unit 31. When step ST2 is started, the biosensor 11 continues acquiring biological information until the power is turned off. Further, the biosensor 11 continues transmitting the acquired biological information to the processing unit 12.

In Embodiment 1, the biosensor 11 is an electrostatic capacity sensor. The biosensor 11 acquires information on electrostatic capacity as biological information. Further, the biosensor 11 transmits the information on the electrostatic capacity to the processing unit 12. The processing unit 12 receives the information on the electrostatic capacity from the biosensor 11, and converts the electrostatic capacity into frequency with the frequency conversion circuit. Further, the processing unit 12 continues the conversion process while receiving the information on the electrostatic capacity from the biosensor 11. Further, the processing unit 12 may continue storing the converted information in the storage unit.

In step ST3, the processing unit 12 outputs biological information.

In Embodiment 1, the processing unit 12 outputs the information converted from electrostatic capacity into frequency. For example, the processing unit 12 transmits information to the calculation unit on the basis of the trigger information for starting measurement. For example, the trigger information for starting measurement is based on the input information of the operation display unit 31. The input information is information indicating whether or not the measurement start button to start measurement has been pressed, for example. The calculation unit may be included in the measurement device 1A or may be included in a device different from the measurement device 1A according to various exemplary aspects.

The calculation unit calculates the amount of the measurement target on the basis of the information received from the processing unit 12. In Embodiment 1, the amount of the measurement target is a moisture amount.

Information on the amount of the measurement target calculated by the calculation unit is transmitted to the operation display unit 31. The operation display unit 31 displays information on the amount of the measurement target.

As described above, by performing steps ST1 to ST3, the biosensor 11 is brought into contact with the measurement portion of a living body, and biological information may be acquired and outputted.

[Using Method of Measurement Device]

An example of a using method of the measurement device 1A will be described. FIG. 6 is a schematic view of an example of a state in which the measurement device 1A of Embodiment 1 according to the present invention is used. Hereinafter, an example of a method of using an intraoral measurement device will be described as an example for the measurement device 1A.

As illustrated in FIG. 6, a cover film 3 covers the sensor portion 10 and the probe portion 20 of the measurement device 1A.

FIG. 7 is a schematic diagram of an example of the cover film 3. As illustrated in FIG. 7, the cover film 3 has a membrane portion 3a that isolates liquid and gas from each other. The membrane portion 3a is a film that does not allow liquid to permeate, but allows gas to permeate in the exemplary aspect. For example, the membrane portion 3a is a hydrophobic air-permeable membrane. The membrane portion 3a is formed in a frame shape as shown in FIG. 7, for example.

Note that the shape of the membrane portion 3a can be varied in accordance with the shape of the detection surface 11a of the biosensor 11 and positions of the multiple suction holes 41. For example, an entire portion of the cover film 3 covering the sensor portion 10 may be formed of the membrane portion 3a. Alternatively, a portion of the cover film 3 covering the contact surface 10a may be formed of the membrane portion 3a.

A portion other than the membrane portion 3a is a film that is impermeable to liquid and gas.

In a state in which the cover film 3 is attached to the measurement device 1A, the membrane portion 3a is disposed at a position where the multiple suction holes 41 are disposed. With this configuration, gas is sucked, but liquid is not sucked into the multiple suction holes 41.

The power button of the operation display unit 31 is pressed to turn on the power supply of the measurement device 1A. With this, the measurement device 1A is brought into a state in which a measurement may be performed.

In the measurement, the contact surface 10a of the measurement device 1A is brought into contact with the measurement portion in the oral cavity of a user. For example, the contact surface 10a is brought into contact with the tongue portion of the user.

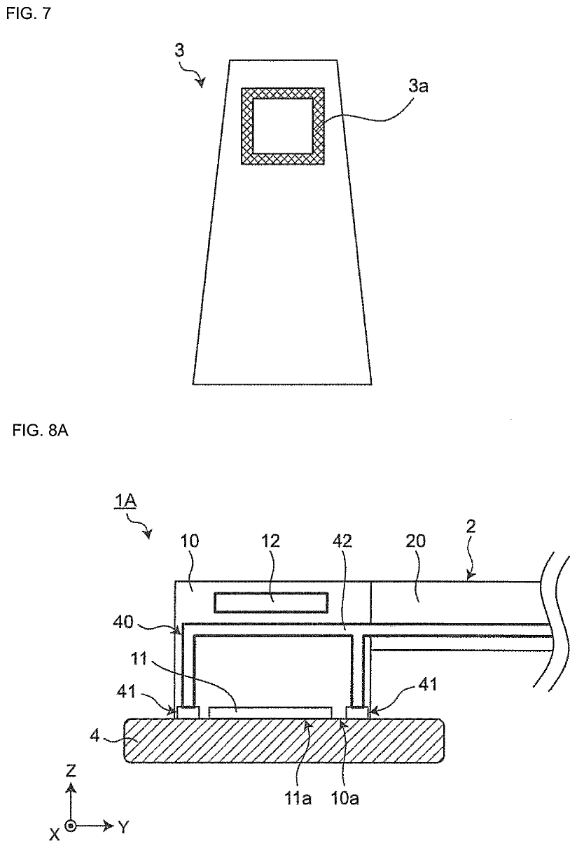

Each of FIG. 8A and FIG. 8B is a schematic diagram of an example of a state in which the measurement device 1A of Embodiment 1 is brought into contact with a living body according to an exemplary aspect. As illustrated in FIG. 8A, the contact surface 10a of the measurement device 1A is brought into contact with a living body 4, that is, the measurement portion in the oral cavity of the user. Next, the suction start button of the operation display unit 31 is pressed to start sucking by the suction portion 40. As illustrated in FIG. 8B, the living body 4 is sucked from the multiple suction holes 41, and the living body 4 comes into contact with the detection surface 11a of the biosensor 11. Further, with the suction force of the multiple suction holes 41, the state in which the living body 4 is in contact with the detection surface 11a of the biosensor 11 may be maintained.

A measurement is started in a state in which the living body 4 is in contact with the detection surface 11a of the biosensor 11. For example, the measurement is started by pressing the measurement start button of the operation display unit 31.

The measurement device 1A performs an example of the operation illustrated in FIG. 5. That is, in the measurement device 1A, when the trigger information for starting measurement is received, the processing unit 12 outputs the information, obtained by converting the biological information acquired by the biosensor 11, to the calculation unit. The calculation unit calculates the amount of the measurement target on the basis of the information from the processing unit 12.

When the measurement is completed, information on the amount of the measurement target is displayed on the operation display unit 31 as the measurement result. For example, the measurement device 1A may include a speaker, and the user may be notified of the end of a measurement by audio information from the speaker in an exemplary aspect.

### Technical Effects

With the use of the measurement device 1A according to Embodiment 1, the following effects may be exhibited.

The measurement device 1A has the contact surface 10a that comes into contact with the measurement portion of a living body. As described above, the measurement device 1A includes the biosensor 11 and the suction portion 40. The biosensor 11 is disposed on the contact surface 10a and has the detection surface 11a that acquires biological information. The suction portion 40 sucks a living body from multiple suction holes 41 provided in the contact surface 10a on the periphery of the detection surface 11a of the biosensor 11.

With the configuration above, the measurement accuracy may be increased. By sucking a living body by the suction portion 40, the detection surface 11a of the biosensor 11 is easily brought into contact with the living body. Further, with the suction force of the suction portion 40, the state in which the detection surface 11a of the biosensor 11 and a living body are in contact with each other may easily be maintained. With this configuration, variation in the measurement value depending on the usage of a user can be suppressed.

Since a living body may be attracted with suction caused by the suction portion 40, even when the measurement portion of the living body has unevenness, the living body may easily be brought into contact with the detection surface 11a of the biosensor 11.

Even when the detection surface 11a of the biosensor 11 is brought into contact with the measurement portion in the oral cavity, the user may easily accomplish the contact without visual confirmation.

The measurement device 1A includes the housing 2 having the longer direction D1. The housing 2 includes the sensor portion 10 and the grip portion 30. The sensor portion 10 is provided on the one end E1 side in the longer direction D1. The grip portion 30 is provided on the other end E2 side in the longer direction D1. The biosensor 11 is disposed in the sensor portion 10. The multiple suction holes 41 are provided to sandwich the biosensor 11 in the longer direction D1.

With the configuration above, the measurement accuracy may further be increased. With the use of the measurement device 1A, the multiple suction holes 41 are provided sandwiching the biosensor 11 in the longer direction D1 of the housing 2. With this configuration, by sucking a living body from the multiple suction holes 41, the living body may easily be brought into contact with the detection surface 11a of the biosensor 11 in the longer direction D1 of the housing 2. For example, floating of the detection surface 11a of the biosensor 11 from the living body in the longer direction D1 may be suppressed. As a result, the measurement accuracy may further be increased.

The multiple suction holes 41 are symmetrically provided with respect to the biosensor 11. With the configuration above, the measurement accuracy may further be increased.

Note that, in Embodiment 1, an example has been described in which the measurement device 1A includes the biosensor 11, the processing unit 12, the operation display unit 31, and the suction portion 40, but the present invention is not limited thereto. In the measurement device 1A, these components may be implemented with one device or may be implemented with multiple devices. For example, the biosensor 11 and the processing unit 12 may integrally be formed. Moreover, the processing unit 12 and the operation display unit 31 may integrally be formed. In addition, the processing unit 12 and the calculation unit may integrally be formed.

In Embodiment 1, an example has been described in which the operation display unit 31 is provided in the measurement device 1A, but the present invention is not limited thereto. The operation display unit 31 is not required to be provided in the measurement device 1A. For example, the operation display unit 31 may be provided in a processing device different from the measurement device 1A in an alternative aspect.

In Embodiment 1, an example has been described in which the measurement target of the measurement device 1A is moisture and the measurement device 1A measures the moisture amount in the oral cavity, but the present invention is not limited thereto. In a case in which the measurement device 1A is an intraoral measurement device, it is sufficient that the measurement device 1A is configured to measure the intraoral state. For example, the measurement device 1A may measure the secretion amount of saliva, occlusal force, lingual pressure, lingual color tone, and/or the amount of various substances contained in saliva. Specifically, the measurement device 1A may measure such as the amount of secreted electrolyte, various enzymes, protein or ammonia as the measurement target.

Alternatively, the measurement device 1A can be a sphygmograph, a pulse oximeter, or a skin moisture meter, in alternative aspects.

In Embodiment 1, an example has been described in which the housing 2 includes the sensor portion 10, the probe portion 20, and the grip portion 30, but the present invention is not limited thereto. It is sufficient that the housing 2 has a longer direction.

In Embodiment 1, an example has been described in which the biosensor 11 is an electrostatic capacity sensor, but the present invention is not limited thereto. It is sufficient that the biosensor 11 is a sensor configured for acquiring biological information. For example, the biosensor 11 may be at least one of an impedance measurement sensor, a load sensor, and a humidity sensor.

In Embodiment 1, an example has been described in which the detection surface 11a of the biosensor 11 is formed in a rectangular shape when viewed from the height direction (i.e., in the Z direction) of the measurement device 1A, but the present invention is not limited thereto. For example, the detection surface 11a of the biosensor 11 can have a polygonal shape, a circular shape, or an elliptical shape when viewed from the height direction (i.e., in the Z direction) of the measurement device 1A.

In Embodiment 1, an example has been described in which the processing unit 12 includes a conversion circuit to convert electrostatic capacity into frequency, but the present invention is not limited thereto. The processing unit 12 may include a circuit to convert the biological information acquired by the biosensor 11 into information other than frequency. Alternatively, the processing unit 12 is not required to include the conversion circuit. In the case above, the processing unit 12 outputs the biological information acquired by the biosensor 11 as it is. That is, the output of the processing unit 12 is the information on the electrostatic capacity.

In Embodiment 1, an example has been described in which the processing unit 12 transmits information to the calculation unit using the input information of the operation display unit 31 as the trigger information for starting measurement, but the present invention is not limited thereto. The processing unit 12 may transmit information to the calculation unit on the basis of information other than the input information of the operation display unit 31. For example, the processing unit 12 may transmit information to the calculation unit on the basis of the contact information between the biosensor 11 and the measurement portion of a living body and/or the suction pressure of the suction portion 40.

Alternatively, the processing unit 12 may continue transmitting information to the calculation unit without depending on the trigger information for starting measurement. In the case above, the calculation unit may receive the trigger information for starting measurement and start a calculation process on the basis of the trigger information. For example, the calculation unit temporarily stores the information transmitted from the processing unit 12 in a cache memory included in the calculation unit. Upon receiving the trigger information for starting measurement, the calculation unit may store the information of before and after the point of time at which the trigger information is received, from the cache memory to the storage unit, and calculate the amount of the measurement target on the basis of the stored information.

In Embodiment 1, an example has been described in which the processing unit 12 is disposed inside the sensor portion 10, but the present invention is not limited thereto. The processing unit 12 may be disposed inside the probe portion 20. Further, the processing unit 12 may be disposed inside the grip portion 30 in another exemplary aspect.

In Embodiment 1, an example has been described in which the operation display unit 31 includes an operation unit and a display unit, but the present invention is not limited thereto. It is sufficient that the operation display unit 31 includes at least either of the operation unit or the display unit.

In Embodiment 1, steps ST1 to ST3 in FIG. 5 have been described as an example of an operation of the measurement device 1A, but the operation is not limited thereto. For example, steps ST1 to ST3 in FIG. 5 may be combined or divided. Alternatively, the flowchart in FIG. 5 may include additional steps. For example, a step of displaying the measurement result on the operation display unit 31 may be added in another exemplary aspect.

In Embodiment 1, an example has been described in which the multiple suction holes 41 are provided in the contact surface 10a, but the present invention is not limited thereto. It is sufficient that, in the contact surface 10a, one or multiple suction holes 41 are provided on the periphery of the detection surface 11a of the biosensor 11.

In Embodiment 1, an example has been described in which the multiple suction holes 41 are formed in a circular shape, but the present invention is not limited thereto. The multiple suction holes 41 may have a shape other than a circle. For example, the multiple suction holes 41 may have an elliptical shape or a polygonal shape.

In Embodiment 1, an example has been described in which the multiple suction holes 41 have the same shape, but the present invention is not limited thereto. Each of the multiple suction holes 41 may have a different shape.

In Embodiment 1, an example has been described in which the multiple suction holes 41 are symmetrically provided with respect to the biosensor 11, but the present invention is not limited thereto. Further, an example has been described in which the multiple suction holes 41 are provided along the axial line CL1 in the longer direction D1 of the housing 2, but the present invention is not limited thereto. The multiple suction holes 41 are not required to be symmetrically provided with respect to the biosensor 11. The multiple suction holes 41 are not required to be provided along the axial line CL1 in the longer direction D1 of the housing 2.

In Embodiment 1, an example has been described in which the multiple suction holes 41 are provided along the longer direction D1 of the housing 2, but the present invention is not limited thereto.

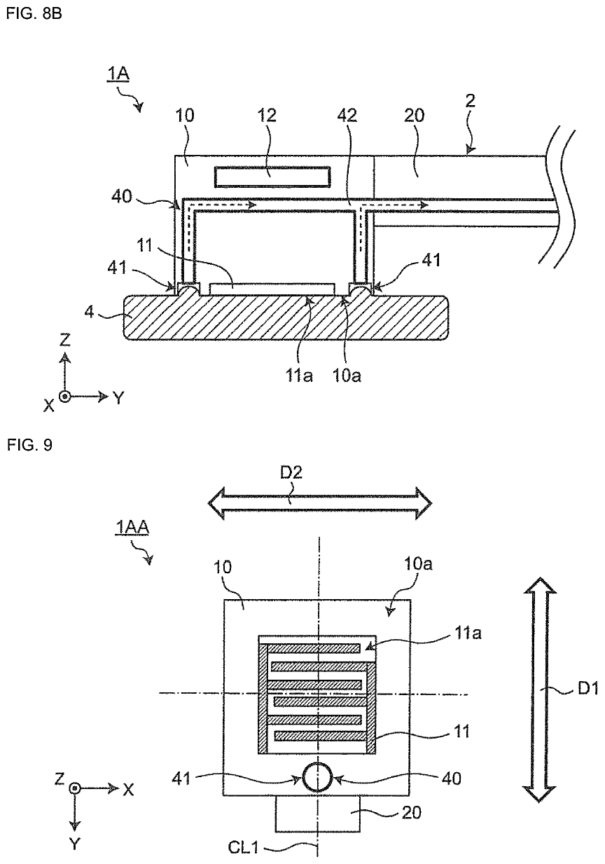

FIG. 9 is a schematic enlarged diagram of part of a measurement device 1AA of a modification of Embodiment 1 according to an exemplary aspect. As illustrated in FIG. 9, the measurement device 1AA is provided with one suction hole 41 in the contact surface 10a. In particular, the suction hole 41 is provided on the side of the probe portion 20, in the contact surface 10a of the sensor portion 10. Even in the configuration above, the living body may be sucked from the suction hole 41, and the measurement accuracy may be increased.

FIG. 10 is a schematic enlarged diagram of part of a measurement device 1AB of a modification of Embodiment 1 according to an exemplary aspect. As illustrated in FIG. 10, multiple suction holes 41a of the measurement device 1AB have a rectangular shape. The multiple suction holes 41a are provided to sandwich the biosensor 11 in the longer direction D1 of the housing 2. Further, the multiple suction holes 41a are provided along both ends of the detection surface 11a of the biosensor 11 by extending in the shorter direction D2 (X direction) of the housing 2. With the configuration above, the suction area of the multiple suction holes 41a may be increased, and thus a living body more easily comes into contact with the detection surface 11a of the biosensor 11. With this configuration, the measurement accuracy can further be increased.

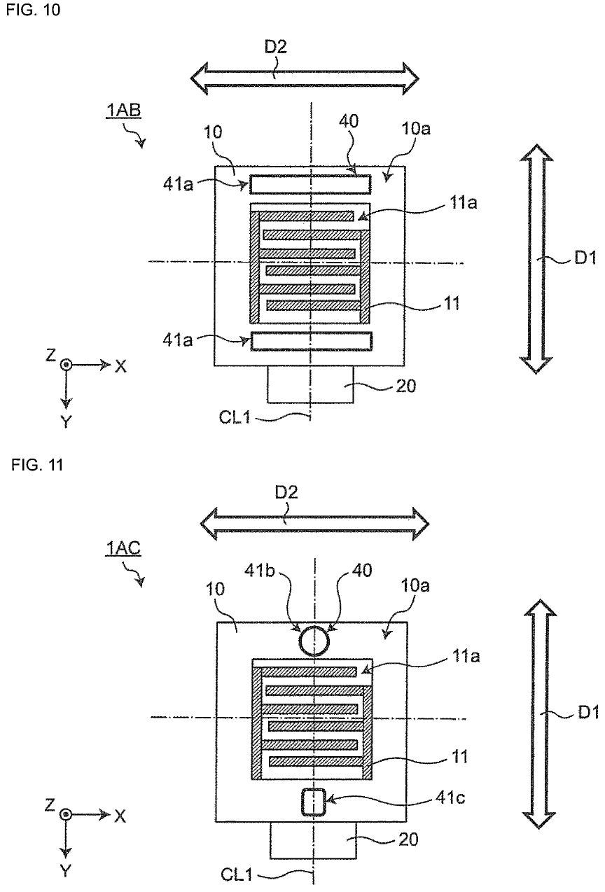

FIG. 11 is a schematic enlarged diagram of part of a measurement device 1AC of a modification of Embodiment 1 according to an exemplary aspect. As illustrated in FIG. 11, multiple suction holes 41b and 41c of the measurement device 1AC respectively have different shapes. In particular, the suction hole 41b has a circular shape. The suction hole 41c has a rectangular shape. Even in the configuration above, the multiple suction holes 41b and 41c may suck a living body, and the measurement accuracy can be increased.

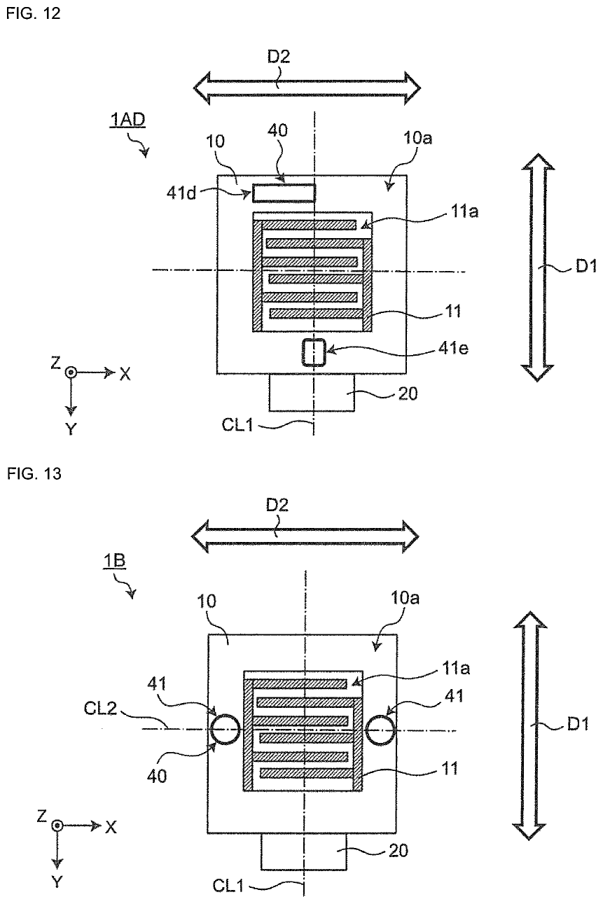

FIG. 12 is a schematic enlarged diagram of part of a measurement device 1AD of a modification of Embodiment 1 according to an exemplary aspect. As illustrated in FIG. 12, multiple suction holes 41d and 41e of the measurement device 1AD are not symmetrically disposed with respect to the biosensor 11. The multiple suction holes 41d and 41e have different shapes. Even in the configuration above, the multiple suction holes 41d and 41e may suck a living body, and the measurement accuracy may be increased.

### Embodiment 2

A measurement device according to Embodiment 2 of an exemplary aspect will be described. Note that, in Embodiment 2, different points from Embodiment 1 will mainly be described. In Embodiment 2, components identical or equivalent to those in Embodiment 1 are denoted by the same reference signs. Further, in Embodiment 2, a description overlapping with Embodiment 1 will be omitted.

An example of a measurement device of Embodiment 2 will be described with reference to FIG. 13. FIG. 13 is a schematic enlarged diagram of part of a measurement device 1B of Embodiment 2 according to the present invention.

As shown, Embodiment 2 is different from Embodiment 1 in that the multiple suction holes 41 are provided to sandwich the biosensor 11 in the shorter direction D2 of the housing 2.

As illustrated in FIG. 13, the multiple suction holes 41 in the measurement device 1B are provided along the shorter direction D2 (i.e., in the X direction) orthogonal to the longer direction D1 of the housing 2. Specifically, the multiple suction holes 41 are provided to sandwich the biosensor 11 in the shorter direction D2 (i.e., in the X direction) orthogonal to the longer direction D1 (i.e., in the Y direction) of the housing 2. That is, the multiple suction holes 41 are provided on both sides of the biosensor 11 in the shorter direction D2 of the housing 2.

In Embodiment 2, the multiple suction holes 41 are provided along an axial line CL2 in the shorter direction D2 of the housing 2 when viewed from the height direction (i.e., in the Z direction) of the measurement device 1B. The axial line CL2 is a line that extends in the shorter direction D2 of the housing 2 and passes through the center of the detection surface 11a of the biosensor 11 when the measurement device 1B is viewed from the side of the contact surface 10a. The axial line CL2 is orthogonal to the axial line CL1.

The multiple suction holes 41 are symmetrically provided with respect to the biosensor 11. Specifically, the multiple suction holes 41 are symmetrically provided with respect to the biosensor 11 in the shorter direction D2 of the housing 2.

### Technical Effects

With the use of the measurement device 1B according to Embodiment 2, the following effects may be exhibited.

The measurement device 1B includes the housing 2 having a longer direction D1. The housing 2 includes the sensor portion 10 and the grip portion 30. The sensor portion 10 is provided on the one end E1 side in the longer direction D1. The grip portion 30 is provided on the other end E2 side in the longer direction D1. The biosensor 11 is disposed in the sensor portion 10. The multiple suction holes 41 are provided to sandwich the biosensor 11 in the shorter direction D2 orthogonal to the longer direction D1.

With the configuration above, the measurement accuracy may be increased. With the use of the measurement device 1B, the detection surface 11a of the biosensor 11 may easily be brought into contact with a living body in the width direction (i.e., in the X direction) of the measurement device 1B.

With the use of the measurement device 1B, the multiple suction holes 41 are provided to sandwich the biosensor 11 in the shorter direction D2 of the housing 2. With this configuration, by sucking a living body from the multiple suction holes 41, the detection surface 11a of the biosensor 11, in the shorter direction D2 of the housing 2, may easily be brought into contact with the living body. For example, floating of the detection surface 11a of the biosensor 11 from the living body in the shorter direction D2 may be suppressed. As a result, the measurement accuracy may further be increased.

For example, in a case in which the measurement portion of a living body is the buccal mucosa in the oral cavity, the measurement may be performed with the gripping direction of the grip portion 30 inclined by 90° compared to a case in which the lingual mucosa is the measurement portion. Even in the case above, the detection surface 11a of the biosensor 11 can easily be brought into contact with the living body, and the measurement accuracy can be increased.

FIG. 14 is a schematic enlarged diagram of part of a measurement device 1BA of a modification of Embodiment 2 according to an exemplary aspect. As illustrated in FIG. 14, multiple suction holes 41f of the measurement device 1BA have a rectangular shape. The multiple suction holes 41f are provided to sandwich the biosensor 11 in the shorter direction D2 of the housing 2. Further, the multiple suction holes 41f are provided along both ends of the detection surface 11a of the biosensor 11 by extending in the longer direction D1 (i.e., in the Y direction) of the housing 2. With the configuration above, the suction area of the multiple suction holes 41f may be increased, and thus the detection surface 11a of the biosensor 11 may more easily be brought into contact with a living body. With this, the measurement accuracy may further be increased.

### Embodiment 3

A measurement device according to Embodiment 3 of an exemplary aspect will be described. Note that, in Embodiment 3, different points from Embodiment 1 and Embodiment 2 will mainly be described. In Embodiment 3, components identical or equivalent to those in Embodiment 1 and Embodiment 2 are denoted by the same reference signs. Further, in Embodiment 3, a description overlapping with Embodiment 1 and Embodiment 2 will be omitted.

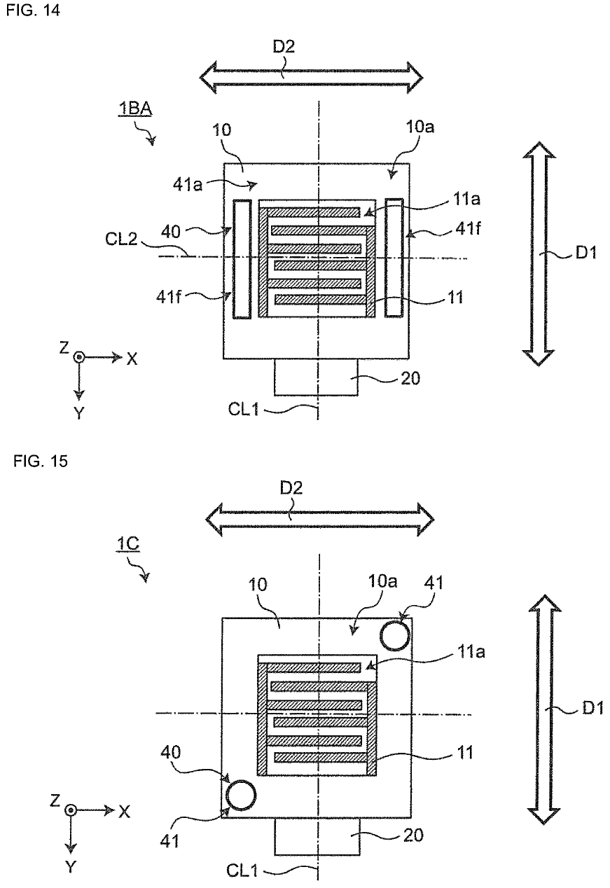

An example of a measurement device of Embodiment 3 will be described with reference to FIG. 15. FIG. 15 is a schematic enlarged diagram of part of a measurement device 1C of Embodiment 3 according to the present invention.

As shown, Embodiment 3 is different from Embodiment 1 and Embodiment 2 in that multiple suction holes 41 are provided in corner portions of the detection surface 11a of the biosensor 11.

As illustrated in FIG. 15, in the measurement device 1C, the multiple suction holes 41 are provided in the corner portions of the detection surface 11a of the biosensor 11. The corner portion means a portion where two adjacent sides, among multiple sides defining the outer periphery of the detection surface 11a having a polygonal shape, intersect and are connected to each other when viewed from the height direction (i.e., in the Z direction) of the measurement device 1C.

In Embodiment 3, the detection surface 11a of the biosensor 11 has a rectangular shape when viewed from the height direction (i.e., in the Z direction) of the measurement device 1C. The detection surface 11a has the four corner portions. In the measurement device 1C, the two suction holes 41 are provided in two corner portions among four corner portions of the detection surface 11a. The two suction holes 41 are symmetrically provided with respect to the biosensor 11. Specifically, the two suction holes 41 are provided on an extension line of a diagonal line of the detection surface 11a having a rectangular shape.

### Technical Effects

With the use of the measurement device 1C according to Embodiment 3, the following effects may be exhibited.

In the measurement device 1C, the detection surface 11a of the biosensor 11 has a polygonal shape. The multiple suction holes 41 are provided in corner portions of the detection surface 11a. With the configuration above, the detection surface 11a of the biosensor 11 may more easily be brought into contact with a living body. With this, the measurement accuracy can further be increased.

Note that, in Embodiment 3, an example has been described in which the detection surface 11a of the biosensor 11 has a rectangular shape when viewed from the height direction (i.e., in the Z direction) of the measurement device 1C, but the present invention is not limited thereto. It is noted that it is sufficient that the detection surface 11a has a shape with a corner portion. For example, it is sufficient that the detection surface 11a has a polygonal shape.

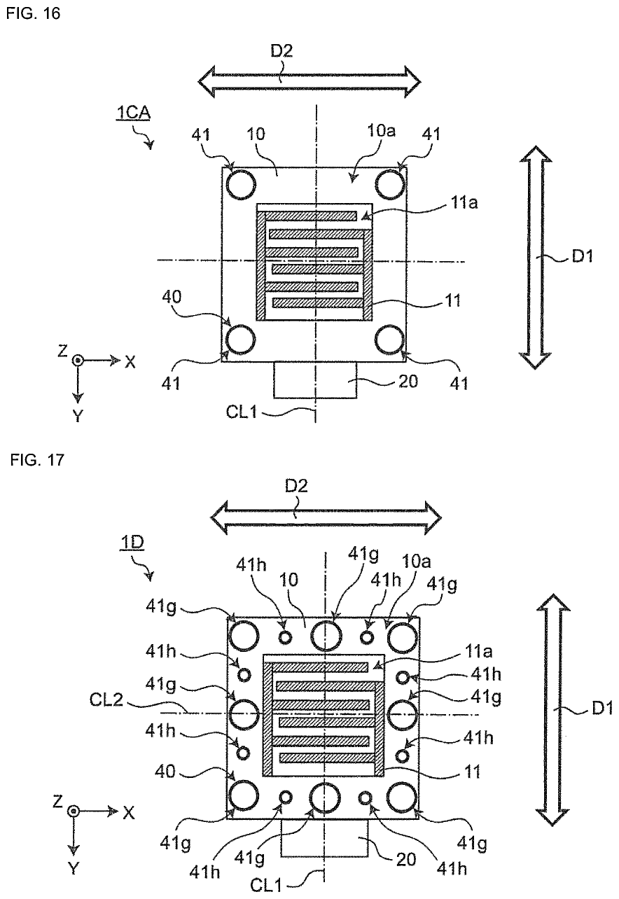

FIG. 16 is a schematic enlarged diagram of part of a measurement device 1CA of a modification of Embodiment 3 according to an exemplary aspect. As illustrated in FIG. 16, the multiple suction holes 41 of the measurement device 1CA are provided in all corner portions of the detection surface 11a of the biosensor 11. Further, the multiple suction holes 41 are symmetrically provided with respect to the biosensor 11. With the configuration above, the measurement accuracy can further be increased.

### Embodiment 4

A measurement device according to Embodiment 4 of an exemplary aspect will be described. Note that, in Embodiment 4, different points from Embodiment 1 to Embodiment 3 will mainly be described. In Embodiment 4, components identical or equivalent to those in Embodiment 1 to Embodiment 3 are denoted by the same reference signs. Further, in Embodiment 4, a description overlapping with Embodiment 1 to Embodiment 3 will be omitted.

An example of a measurement device of Embodiment 4 will be described with reference to FIG. 17. FIG. 17 is a schematic enlarged diagram of part of a measurement device 1D of Embodiment 4 according to an exemplary aspect.

Embodiment 4 is different from Embodiment 1 to Embodiment 3 in that multiple first suction holes 41g and multiple second suction holes 41h having different opening areas are provided in the contact surface 10a, and in that the multiple first suction holes 41g and the multiple second suction holes 41h are provided to surround the periphery of the detection surface 11a of the biosensor 11.

As illustrated in FIG. 17, in the measurement device 1D, the multiple first suction holes 41g and the multiple second suction holes 41h are provided in the contact surface 10a. The multiple first suction holes 41g and the multiple second suction holes 41h are provided to surround the periphery of the detection surface 11a of the biosensor 11. The multiple first suction holes 41g and the multiple second suction holes 41h have different opening areas. Specifically, the opening area of the multiple first suction holes 41g is smaller than the opening area of the multiple second suction holes 41h. Note that the opening area means an area of each of the suction holes 41g and 41h when the measurement device 1D is viewed from the Z direction.

The multiple first suction holes 41g are provided at positions where the detection surface 11a of the biosensor 11 is less likely to come into contact with a living body relative to positions where the multiple second suction holes 41h are provided. For example, the multiple first suction holes 41g may be provided in corner portions of the detection surface 11a of the biosensor 11. Further, the multiple first suction holes 41g may be provided along the axial line CL1 extending in the longer direction D1 of the housing 2 and passing through the center of the detection surface 11a. Moreover, the multiple first suction holes 41g may be provided along the axial line CL2 extending in the shorter direction D2 of the housing 2 and passing through the center of the detection surface 11a.

The multiple second suction holes 41h are provided at positions other than the positions where the multiple first suction holes 41g are provided. For example, the multiple second suction holes 41h are provided between the multiple first suction holes 41g.

### Technical Effects

With the use of the measurement device 1D according to Embodiment 4, the following effects may be exhibited.

In the measurement device 1D, the multiple first suction holes 41g and the multiple second suction holes 41h are provided in the contact surface 10a. The multiple first suction holes 41g and the multiple second suction holes 41h are provided to surround the periphery of the detection surface 11a of the biosensor 11.

With the configuration above, the detection surface 11a of the biosensor 11 may more easily be brought into contact with a living body. Further, by increasing the suction area, the detection surface 11a of the biosensor 11 may more easily be brought into contact with a living body. Moreover, the state in which the detection surface 11a of the biosensor 11 is brought into contact with a living body may easily be maintained. As a result, the measurement accuracy can further be increased.

The multiple first suction holes 41g and the multiple second suction holes 41h have different opening areas. With this, the suction force of suction from the multiple first suction holes 41g is different from the suction force of suction from the multiple second suction holes 41h. Specifically, the opening area of the multiple first suction holes 41g is larger than the opening area of the multiple second suction holes 41h. With this, the suction force of suction from the multiple first suction holes 41g may be made larger than the suction force of suction from the multiple second suction holes 41h.

For example, by providing the multiple first suction holes 41g at positions where the detection surface 11a of the biosensor 11 is less likely to come into contact with a living body relative to positions where the multiple second suction holes 41h are provided, the detection surface 11a of the biosensor 11 may more easily be brought into contact with the living body. With this, the measurement accuracy can further be increased.

Note that, in Embodiment 4, an example has been described in which the multiple first suction holes 41g and the multiple second suction holes 41h having different opening areas are provided in the contact surface 10a, but the present invention is not limited thereto. In the measurement device 1D, the opening areas of the multiple suction holes 41g and 41h may be the same in an alternative aspect.

### Embodiment 5

A measurement device according to Embodiment 5 of an exemplary aspect will be described. Note that, in Embodiment 5, different points from Embodiment 1 to Embodiment 4 will mainly be described. In Embodiment 5, components identical or equivalent to those in Embodiment 1 to Embodiment 4 are denoted by the same reference signs. Further, in Embodiment 5, a description overlapping with Embodiment 1 to Embodiment 4 will be omitted.

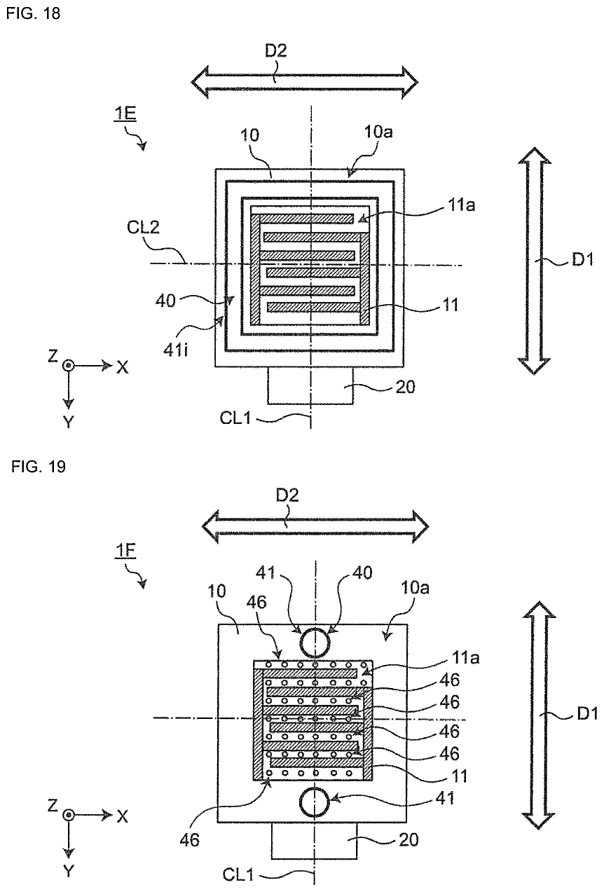

An example of a measurement device of Embodiment 5 will be described with reference to FIG. 18. FIG. 18 is a schematic enlarged diagram of part of a measurement device 1E of Embodiment 5 according to the present invention.

Embodiment 5 is different from Embodiment 1 to Embodiment 4 in that a frame-shaped suction hole 41i is provided in the contact surface 10a.

As illustrated in FIG. 18, in the measurement device 1E, one frame-shaped suction hole 41i is provided in the contact surface 10a. The outer shape of the suction hole 41i has a rectangular shape. The detection surface 11a of the biosensor 11 is disposed within the frame of the suction hole 41i. That is, the detection surface 11a of the biosensor 11 is surrounded by the suction hole 41i.

### Technical Effects

With the use of the measurement device 1E according to Embodiment 5, the following effects may be exhibited.

In the measurement device 1E, the suction hole 41i has a frame shape. The detection surface 11a of the biosensor 11 is disposed inside the frame-shaped suction hole 41i. With the configuration above, the measurement accuracy may further be increased. Further, a living body may be sucked from the suction hole 41i with more uniform suction force.

Note that, in Embodiment 5, an example has been described in which the outer shape of the suction hole 41i is rectangular, but the present invention is not limited thereto. The outer shape of the suction hole 41i may be changed in accordance with the shape of the detection surface 11a of the biosensor 11. For example, the outer shape of the suction hole 41i may have a circular shape, an elliptical shape, or a polygonal shape.

### Embodiment 6

A measurement device according to Embodiment 6 of an exemplary aspect will be described. Note that, in Embodiment 6, different points from Embodiment 1 to Embodiment 5 will mainly be described. In Embodiment 6, components identical or equivalent to those in Embodiment 1 to Embodiment 5 are denoted by the same reference signs. Further, in Embodiment 6, a description overlapping with Embodiment 1 to Embodiment 5 will be omitted.

An example of a measurement device of Embodiment 6 will be described with reference to FIG. 19. FIG. 19 is a schematic enlarged diagram of part of a measurement device 1F of Embodiment 6 according to an exemplary aspect.

Embodiment 6 is different from Embodiment 1 to Embodiment 5 in that multiple sensor suction holes 46 are provided in the detection surface 11a of the biosensor 11.

As illustrated in FIG. 19, in the measurement device 1F, the multiple sensor suction holes 46 are provided in the detection surface 11a of the biosensor 11. A comb-shaped electrode is disposed on the detection surface 11a of the biosensor 11. The multiple sensor suction holes 46 are provided in a region where the comb-shaped electrode is not disposed on the detection surface 11a.

Specifically, in Embodiment 6, two comb-shaped electrodes are disposed with a distance from each other on the detection surface 11a of the biosensor 11. The multiple sensor suction holes 46 are provided between the two comb-shaped electrodes.

The suction portion 40 is configured to suck a living body from the multiple sensor suction holes 46 provided in the detection surface 11a of the biosensor 11, in addition to the multiple suction holes 41 provided on the periphery of the detection surface 11a of the biosensor 11.

The multiple sensor suction holes 46 are connected to the suction path 42. Thus, the pump 43 can suck gas from the multiple sensor suction holes 46 via the suction path 42. With this, a living body may be sucked from the multiple sensor suction holes 46 also in the detection surface 11a of the biosensor 11.

The multiple sensor suction holes 46, except for being provided in the detection surface 11a of the biosensor 11, may have the same shape as the suction holes of Embodiment 1 to Embodiment 5.

### Technical Effects

With the use of the measurement device 1F according to Embodiment 6, the following effects may be exhibited.

In the measurement device 1F, the suction portion 40 sucks a living body from the multiple sensor suction holes 46 provided in the detection surface 11a of the biosensor 11. With the configuration above, the detection surface 11a of the biosensor 11 may more easily be brought into contact with the living body. Further, the detection surface 11a of the biosensor 11 may be brought into uniform contact with the living body. With this, the measurement accuracy can further be increased.

Note that, in Embodiment 6, an example has been described in which the multiple sensor suction holes 46 are provided in the detection surface 11a of the biosensor 11, but the present invention is not limited thereto. In the measurement device 1F, it is sufficient that one or multiple sensor suction holes 46 are provided in the detection surface 11a of the biosensor 11.

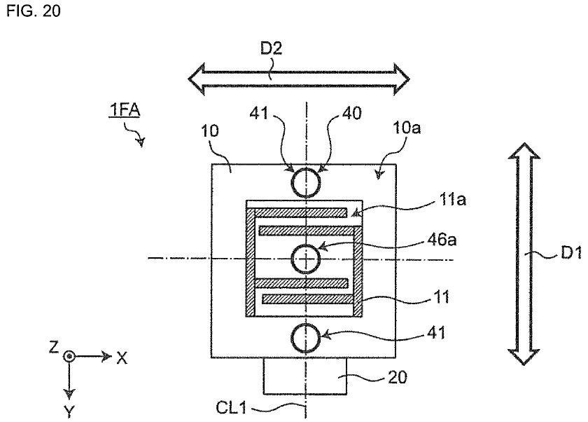

FIG. 20 is a schematic enlarged diagram of part of a measurement device 1FA of a modification of Embodiment 6 according to an exemplary aspect. As illustrated in FIG. 20, in the measurement device 1FA, one sensor suction hole 46a is provided in the detection surface 11a of the biosensor 11. The sensor suction hole 46a is provided at the center of the detection surface 11a when the measurement device 1FA is viewed from the height direction (i.e., in the Z direction). Even in the configuration above, the detection surface 11a of the biosensor 11 may be brought into uniform contact with a living body. With this, the measurement accuracy can further be increased.

### Embodiment 7

A measurement device according to Embodiment 7 of an exemplary aspect will be described. Note that, in Embodiment 7, different points from Embodiment 1 will mainly be described. In Embodiment 7, components identical or equivalent to those in Embodiment 1 are denoted by the same reference signs. Further, in Embodiment 7, a description overlapping with Embodiment 1 will be omitted.

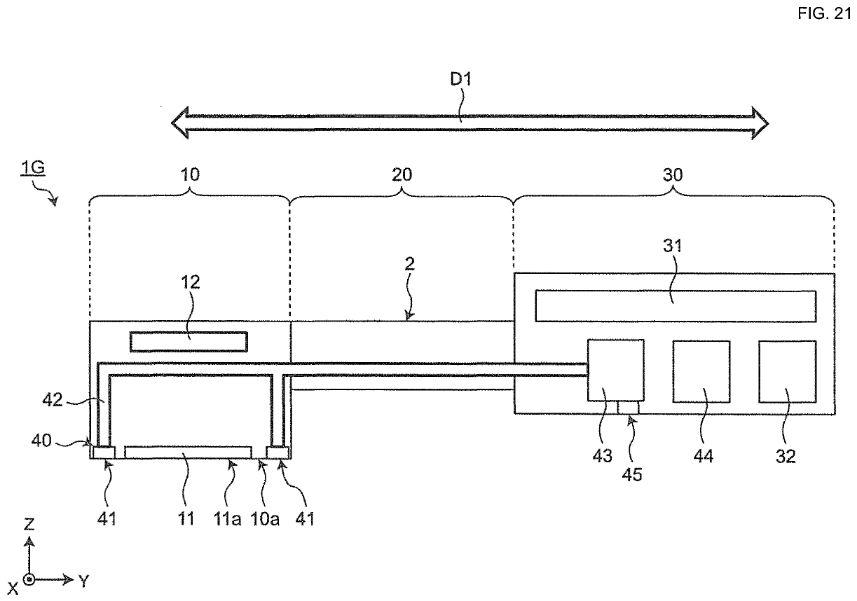

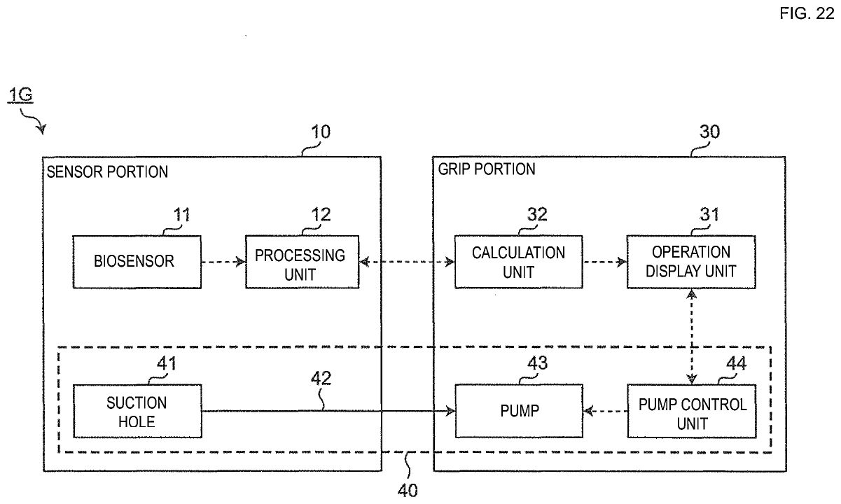

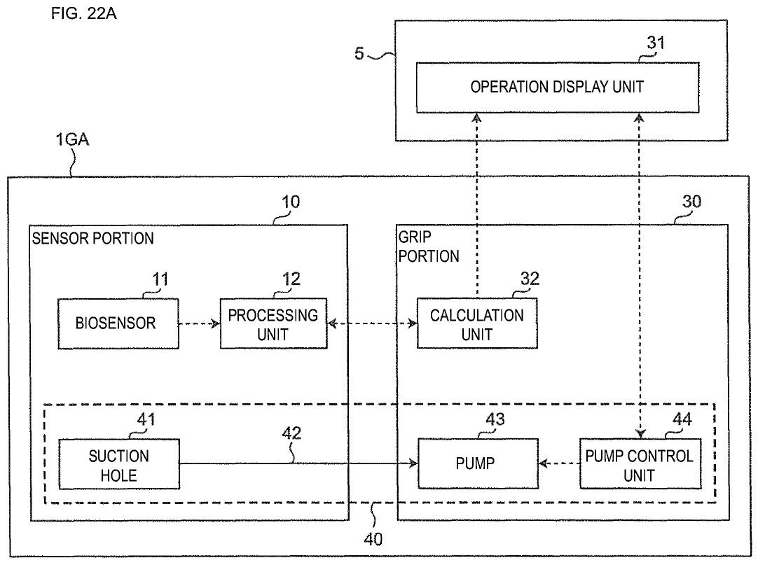

An example of a measurement device of Embodiment 7 will be described with reference to FIG. 21 and FIG. 22. FIG. 21 is a schematic diagram of an internal configuration of an example of a measurement device 1G of Embodiment 7 according to the present invention. FIG. 22 is a block diagram of a schematic configuration of an example of the measurement device 1G of Embodiment 7 according to the exemplary aspect.

Embodiment 7 is different from Embodiment 1 in that a calculation unit 32 is included.

As illustrated in FIG. 21 and FIG. 22, the measurement device 1G includes the calculation unit 32. The calculation unit 32 is configured to calculate the amount of the measurement target on the basis of the biological information acquired by the biosensor 11.

The calculation unit 32 is housed in the grip portion 30 of the housing 2.

The calculation unit 32 may be implemented with such as a semiconductor element. The function of the calculation unit 32 may be formed only by hardware or may be implemented with a combination of hardware and software. The calculation unit 32 includes a moisture amount calculation circuit that calculates the moisture amount on the basis of the change amount of frequency, for example. Note that the change amount of frequency is a difference between a reference frequency and the frequency converted on the basis of the information on the electrostatic capacity in the processing unit 12. The reference frequency means the frequency in typical air atmosphere.

The calculation unit 32 includes a storage unit in the exemplary aspect. The storage unit may be implemented with a hard disk (HDD), an SSD, a RAM, a DRAM, a ferroelectric memory, a flash memory, a magnetic disk, or a combination thereof, for example.

The biological information acquired by the biosensor 11 is converted by the processing unit 12. The calculation unit 32 is configured to calculate the amount of the measurement target on the basis of the information converted by the processing unit 12.

In Embodiment 7, the measurement device 1G measures the moisture amount in the oral cavity as the amount of the measurement target. For example, the biological information acquired by the biosensor 11 is electrostatic capacity. The processing unit 12 converts the electrostatic capacity into frequency information, and transmits the frequency information to the calculation unit 32. The calculation unit 32 calculates the moisture amount on the basis of the frequency information.

While continuing receiving the biological information from the biosensor 11, the processing unit 12 converts the biological information and continues transmitting the converted information to the calculation unit 32. That is, the calculation unit 32 temporarily stores the information transmitted from the processing unit 12 in a cache memory. The calculation unit 32 starts the calculation process on the basis of the trigger information for starting measurement. That is, the calculation unit 32 continues receiving information from the processing unit 12, but does not start calculating the amount of the measurement target unless the trigger information is received. The trigger information for starting measurement may be generated on the basis of contact information between the biosensor 11 and the measurement portion of a living body, the suction pressure of the suction portion 40, and/or the input information inputted to the operation display unit 31, for example.

For example, when the calculation unit 32 receives the trigger information for starting measurement, the calculation unit 32 reads out, from the cache memory, the information received from the processing unit 12 at a time before and after the point of time at which the trigger information is received, and stores the information in the storage unit. The calculation unit 32 calculates the amount of the measurement target on the basis of the information stored in the storage unit.

Information on the amount of the measurement target calculated by the calculation unit 32 is transmitted to the operation display unit 31.

For example, the calculation unit 32 is controlled by a control unit included in the measurement device 1G.

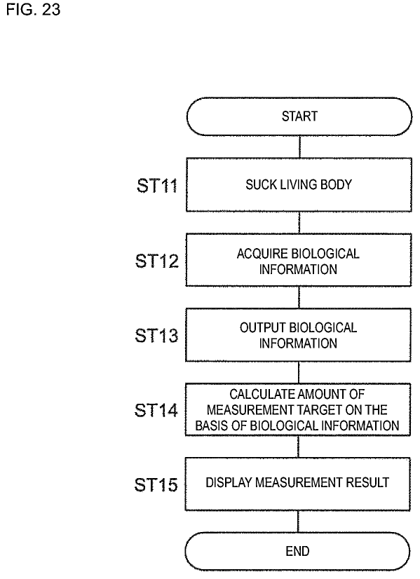

FIG. 23 is a flowchart of an example of an operation of the measurement device 1G of Embodiment 7 according to the present invention. Since steps ST11 to ST12 in FIG. 23 are the same as steps ST1 to ST2 in FIG. 5 of Embodiment 1, detailed description thereof will be omitted.

As illustrated in FIG. 23, in step ST11, the suction portion 40 sucks a living body.

In step ST12, the biosensor 11 acquires biological information.

In step ST13, the processing unit 12 outputs the biological information to the calculation unit 32. The processing unit 12 converts the biological information acquired by the biosensor 11, and transmits the converted information to the calculation unit 32. While continuing receiving the biological information from the biosensor 11, the processing unit 12 converts the biological information and continues transmitting the converted information to the calculation unit 32 (e.g., either continuously or periodically). The calculation unit 32 temporarily stores the information transmitted from the processing unit 12 in the cache memory.

In step ST14, the calculation unit 32 calculates the amount of the measurement target on the basis of the biological information. The calculation unit 32 starts the calculation process on the basis of the trigger information for starting measurement. For example, when the calculation unit 32 receives the trigger information for starting measurement, the calculation unit 32 reads out, from the cache memory, the information received from the processing unit 12 at a time before and after the point of time at which the trigger information is received, and stores the information in the storage unit. The calculation unit 32 calculates the amount of the measurement target based on the information stored in the storage unit and transmits the information on the calculated amount of the measurement target to the operation display unit 31.

In step ST15, the measurement result is displayed by the operation display unit 31. The operation display unit 31 receives the information on the amount of the measurement target from the calculation unit 32, and displays the information.

As described above, by performing steps ST11 to ST15, the measurement device 1G may calculate the amount of the measurement target.

[Technical Effects]

With the use of the measurement device 1G according to Embodiment 7, the following effects may be exhibited.

The measurement device 1G includes the calculation unit 32 that calculates the amount of the measurement target on the basis of the biological information acquired by the biosensor 11. With the configuration above, the amount of the measurement target may be calculated.

Note that, in Embodiment 7, an example has been described in which the calculation unit 32 starts the calculation process on the basis of the trigger information for starting measurement, but the present invention is not limited thereto. The calculation unit 32 may start the calculation process without depending on the trigger information.

In Embodiment 7, an example has been described in which the calculation unit 32 is disposed inside the grip portion 30, but the present invention is not limited thereto. For example, the calculation unit 32 may be disposed inside the sensor portion 10 or the probe portion 20 in alternative aspects. On the other hand, the processing unit 12 may be disposed inside the grip portion 30, in parallel with the calculation unit 32 or in a configuration being included in the calculation unit 32.

In Embodiment 7, an example has been described in which the calculation unit 32 calculates the moisture amount as the amount of the measurement target, but the present invention is not limited thereto. Further, an example has been described in which the calculation unit 32 includes the moisture amount calculation circuit that calculates the moisture amount on the basis of the frequency, but the present invention is not limited thereto. It is sufficient that the calculation unit 32 includes a calculation circuit that calculates the amount of the measurement target.

In Embodiment 7, an example has been described in which the measurement device 1G includes the operation display unit 31, but the present invention is not limited thereto. The measurement device 1G is not required to include the operation display unit 31. For example, the operation display unit 31 may be included in a device different from the measurement device 1G in an alternative aspect.

FIG. 22A is a block diagram of a schematic configuration of a measurement device 1GA of a modification of Embodiment 7 according to an exemplary aspect. FIG. 22A illustrates an example in which the operation display unit 31 is provided in an external device 5 different from the measurement device 1GA. For example, the external device 5 is a device including a display screen and/or an operation unit. Examples of the external device 5 include such as a computer, a display, a touch panel, and a smartphone.

As illustrated in FIG. 22A, the measurement device 1GA may transmit the information calculated by the calculation unit 32 to the operation display unit 31 of the external device 5. With this, the measurement result may be displayed on the operation display unit 31 of the external device 5. Further, in the external device 5, the operation display unit 31 may transmit the inputted input information to the pump control unit 44. The pump control unit 44 may receive the input information from the operation display unit 31 of the external device 5 and control the pump 43 on the basis of the received input information.

For example, the measurement device 1GA and the external device 5 may include a communication unit and communicate with each other via the communication unit. The communication unit includes a circuit that communicates in conformity with a predetermined communication standard. The predetermined communication standard includes LAN, Wi-Fi®, Bluetooth®, USB, HDMI®, controller area network (CAN), serial peripheral interface (SPI), universal asynchronous receiver/transmitter (UART), and inter-integrated circuit (I2C), for example.

### Embodiment 8

A measurement device according to Embodiment 8 of an exemplary aspect will be described. Note that, in Embodiment 8, different points from Embodiment 7 will mainly be described. In Embodiment 8, components identical or equivalent to those in Embodiment 7 are denoted by the same reference signs. Further, in Embodiment 8, a description overlapping with Embodiment 7 will be omitted.

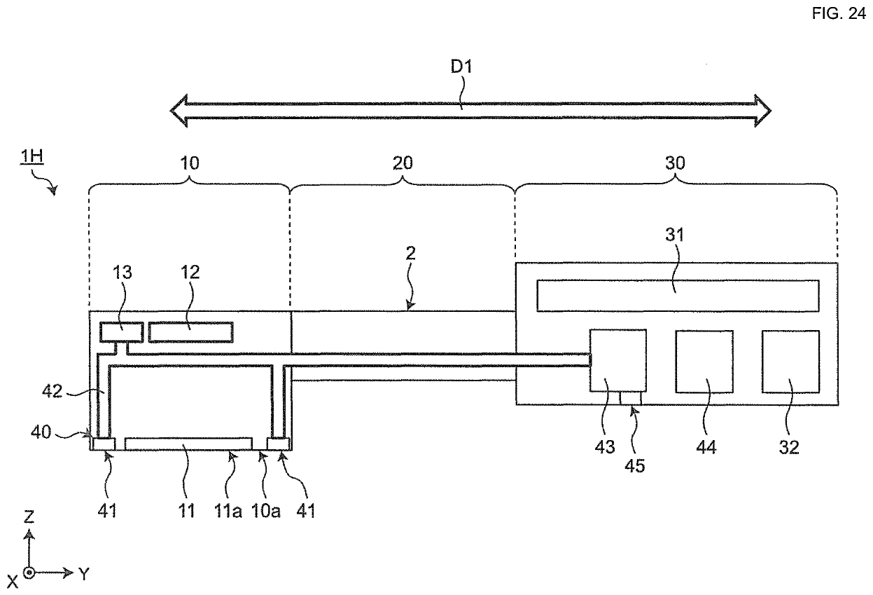

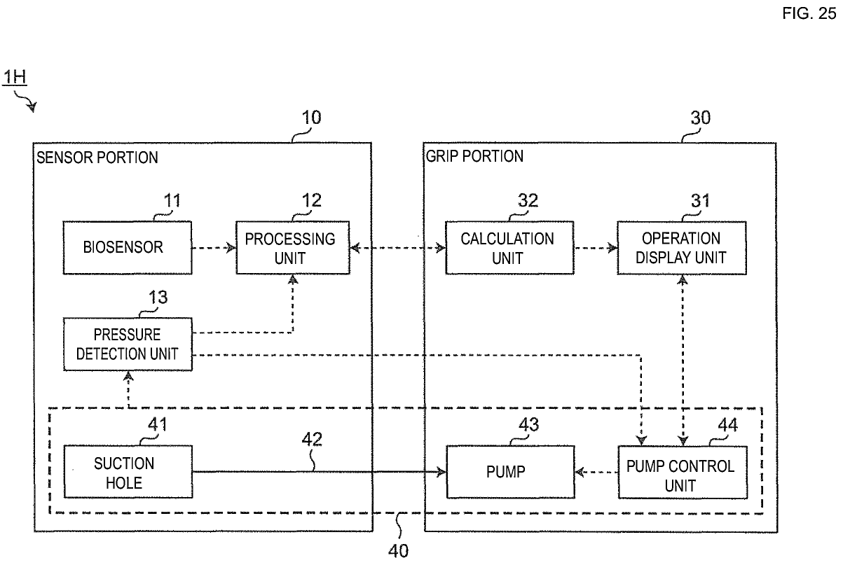

An example of a measurement device of Embodiment 8 will be described with reference to FIG. 24 and FIG. 25. FIG. 24 is a schematic diagram of an internal configuration of an example of a measurement device 1H of Embodiment 8 according to the exemplary aspect. FIG. 25 is a block diagram of a schematic configuration of an example of the measurement device 1H of Embodiment 8 according to the exemplary aspect.

Embodiment 8 is different from Embodiment 7 in that a pressure detection unit 13 is included.

As illustrated in FIG. 24 and FIG. 25, the measurement device 1H includes the pressure detection unit 13. In operation, the pressure detection unit 13 is configured to detect the suction pressure P1 with which the suction portion 40 sucks a living body. For example, the pressure detection unit 13 is a pressure sensor or a differential pressure sensor in exemplary aspects.

The pressure detection unit 13 is disposed inside the sensor portion 10. For example, the pressure detection unit 13 is connected to the suction path 42. The pressure detection unit 13 detects the pressure in the suction path 42 as the suction pressure P1. Note that the pressure detection unit 13 may be disposed in the grip portion 30 instead of the sensor portion 10.

Information on the suction pressure P1 detected by the pressure detection unit 13 is transmitted to the processing unit 12 and the pump control unit 44.

The processing unit 12 also generates trigger information for starting measurement on the basis of the information on the suction pressure P1.

For example, the pressure detection unit 13 is controlled by a control unit included in the measurement device 1H.

The processing unit 12 receives the information on the suction pressure P1 from the pressure detection unit 13, and outputs the trigger information for starting measurement on the basis of the suction pressure P1. Specifically, the processing unit 12 transmits the trigger information to the calculation unit 32.

Moreover, in this exemplary aspect, the pump control unit 44 is configured to control the output of the pump 43 based on the information on the suction pressure P1. For example, the pump control unit 44 controls the output of the pump 43 such that the suction pressure P1 becomes a value appropriate for the measurement. Alternatively, the pump control unit 44 may stop the pump 43 when the suction pressure P1 is lower than a threshold value for a predetermined period.

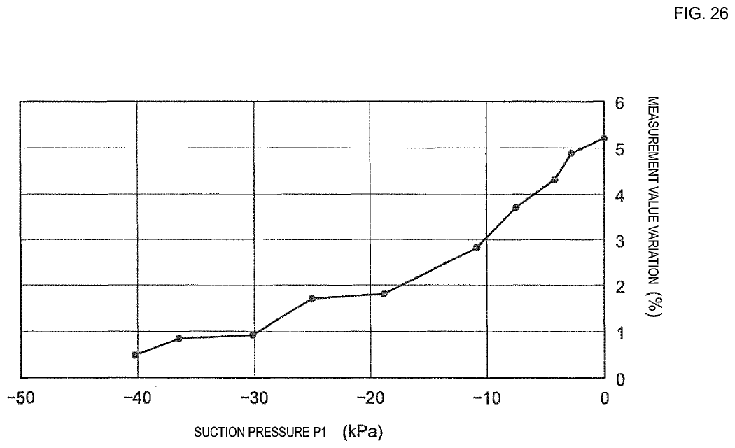

FIG. 26 is a graph of an example of a relationship between the suction pressure P1 and the measurement value variation. As shown, the measurement value variation in FIG. 26 is the variation of the measurement value converted by the processing unit 12. As illustrated in FIG. 26, as the suction pressure P1 increases, the measurement value variation decreases. For example, in a medical site, the measurement value variation is preferably 3% or less. For this reason, the suction pressure P1 is preferably 10 kPa or more and 40 kPa or less. With this, the measurement accuracy can be increased.

According to a guideline for diagnosing oral hypofunction, the inspection is performed by measuring the degree of mucosal wetness in the center part of the dorsum of tongue at approximately 10 mm from the apex of the tongue. At this time, the operation described below is performed. The measurement is performed three times, and the evaluation is performed using a median value to exclude a case that an outlier occurs, thereby increasing the validity of the inspection. On the other hand, when the outlier occurs twice consecutively, there is a possibility that the outlier cannot be excluded even in the operation described above. Note that the outlier means a value of a measurement result with low reliability. As a result of our verification on the issue, it has been found that the outlier is likely to occur in a device having a CV value of 4.5% as measurement accuracy, and as the cause thereof, the contact quality between the tongue and the sensor surface has a insufficient specification. On the other hand, in a device in which the CV value is less than 4.5% and 3.0% or less, it has been found that the contact quality between the tongue and the sensor surface is good and the adequacy of the measurement result is high. Then, under the guideline described above, it is appropriate to set the threshold value to 3.0% in terms of the CV value as the target specification for the device development to increase the adequacy of the measurement result.

More preferably, the suction pressure P1 is 20 kPa or more and 40 kPa or less. With this, since the measurement value variation may be lowered to 2% or less, the measurement accuracy may further be increased.

Note that, by setting the suction pressure P1 to 40 kPa or less, posing damage to a living body due to the suction by the suction portion 40 may be suppressed.

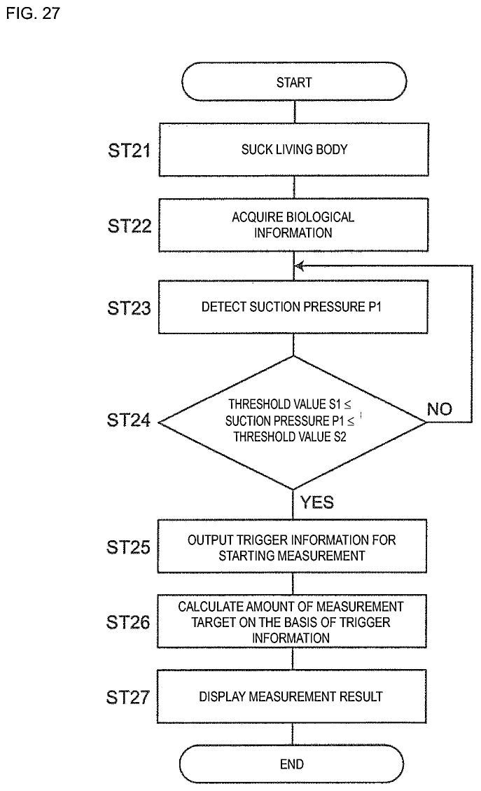

FIG. 27 is a flowchart of an example of an operation of the measurement device 1H of Embodiment 8 according to an exemplary aspect. Since steps ST21 to ST22 in FIG. 27 are the same as steps ST11 to ST12 in FIG. 23 of Embodiment 7, detailed description thereof will be omitted.

As illustrated in FIG. 27, in step ST21, the suction portion 40 sucks a living body.

In step ST22, the biosensor 11 acquires biological information that is transmitted to the processing unit 12. The processing unit 12 converts the biological information and transmits the converted information to the calculation unit 32. The calculation unit 32 temporarily stores the information transmitted from the processing unit 12 in the cache memory.

In step ST23, the suction pressure P1 is detected by the pressure detection unit 13 that detects the pressure in the suction path 42 as the suction pressure P1. Information on the suction pressure P1 detected by the pressure detection unit 13 is transmitted to the processing unit 12 and the pump control unit 44.

The pump control unit 44 controls the output of the pump 43 on the basis of the information on the suction pressure P1. For example, the pump control unit 44 controls the output of the pump 43 such that the suction pressure P1 falls within a predetermined range.

In step ST24, the processing unit 12 determines whether or not the suction pressure P1 is within a predetermined range. The processing unit 12 determines whether or not the suction pressure P1 is in the range of the first threshold value S1 or more and the second threshold value S2 or less. Preferably, the first threshold value S1 is 10 kPa and the second threshold value S2 is 40 kPa. More preferably, the first threshold value S1 is 20 kPa and the second threshold value S2 is 40 kPa.

When the processing unit 12 determines in step ST24 that the suction pressure P1 is in the range of the first threshold value S1 or more and the second threshold value S2 or less, the flow proceeds to step ST25. When the processing unit 12 determines that the suction pressure P1 is out of the range of the first threshold value S1 or more and the second threshold value or less, the flow returns to step ST23.

Note that the suction pressure P1 is used for the determination in step ST24, but the present invention is not limited thereto. The determination in step ST24 may use a mean value, a median value, a minimum value, or a maximum value of the suction pressure P1.

In step ST25, the processing unit 12 generates trigger information for starting measurement. The processing unit 12 transmits the trigger information to the calculation unit 32.

In step ST26, the calculation unit 32 calculates the amount of the measurement target on the basis of the trigger information. The calculation unit 32 receives the trigger information from the processing unit 12 and/or the pump control unit 44. The calculation unit 32 reads out, from the cache memory, the information based on the biological information received from the processing unit 12 at a time before and after the point of time at which the trigger information is received, and stores the information in the storage unit. The calculation unit 32 calculates the amount of the measurement target on the basis of the information stored in the storage unit. The calculation unit 32 transmits the information on the calculated amount of the measurement target to the operation display unit 31.

In step ST27, the measurement result is displayed on the operation display unit 31. The operation display unit 31 receives the information on the amount of the measurement target from the calculation unit 32, and displays the information.

As described above, by performing steps ST21 to ST27, the measurement device 1H may start the measurement process on the basis of on the suction pressure P1 and calculate the amount of the measurement target.

### Technical Effects

With the use of the measurement device 1H according to Embodiment 8, the following effects may be exhibited.

The measurement device 1H includes the pressure detection unit 13 that detects a suction pressure P1 with which the suction portion 40 suctions a living body. The processing unit 12 outputs the trigger information for starting measurement on the basis of the suction pressure P1 detected by the pressure detection unit 13. With the configuration above, the measurement process can be started based on the suction pressure P1 and the amount of the measurement target can then be calculated.

Further, with the use of the measurement device 1H, it is possible to calculate the amount of the measurement target on the basis of the biological information with the suction pressure P1 being appropriate for the measurement, and thus, measurement value variation may be suppressed. With this configuration, the measurement accuracy can be increased.

Note that, in Embodiment 8, an example has been described in which the measurement device 1H includes the calculation unit 32, but the present invention is not limited thereto. For example, the calculation unit 32 may be included in a device different from the measurement device 1H.

In Embodiment 8, an example has been described in which the pressure detection unit 13 is disposed in the sensor portion 10, but the present invention is not limited thereto. For example, the pressure detection unit 13 may be disposed in the probe portion 20 or the grip portion 30.

In Embodiment 8, an example has been described in which information on the suction pressure P1 detected by the pressure detection unit 13 is transmitted to the processing unit 12 and the pump control unit 44, but the present invention is not limited thereto. For example, information on the suction pressure P1 detected by the pressure detection unit 13 may be transmitted to the processing unit 12 and/or the calculation unit 32. In the case above, the processing executed by the processing unit 12 described in Embodiment 8 may be executed by the processing unit 12 and/or the calculation unit 32.

In Embodiment 8, an example has been described in which the pump control unit 44 controls the output of the pump 43 on the basis of the information on the suction pressure P1 detected by the pressure detection unit 13, but the present invention is not limited thereto. The pump control unit 44 may control the output of the pump 43 without depending on the information on the suction pressure P1 detected by the pressure detection unit 13. For example, the pump control unit 44 may control the pump 43 such that the output of the pump 43 falls within a predetermined range.

### Embodiment 9

A measurement device according to Embodiment 9 of an exemplary aspect will be described. Note that, in Embodiment 9, different points from Embodiment 8 will mainly be described. In Embodiment 9, components identical or equivalent to those in Embodiment 8 are denoted by the same reference signs. Further, in Embodiment 9, a description overlapping with Embodiment 8 will be omitted.

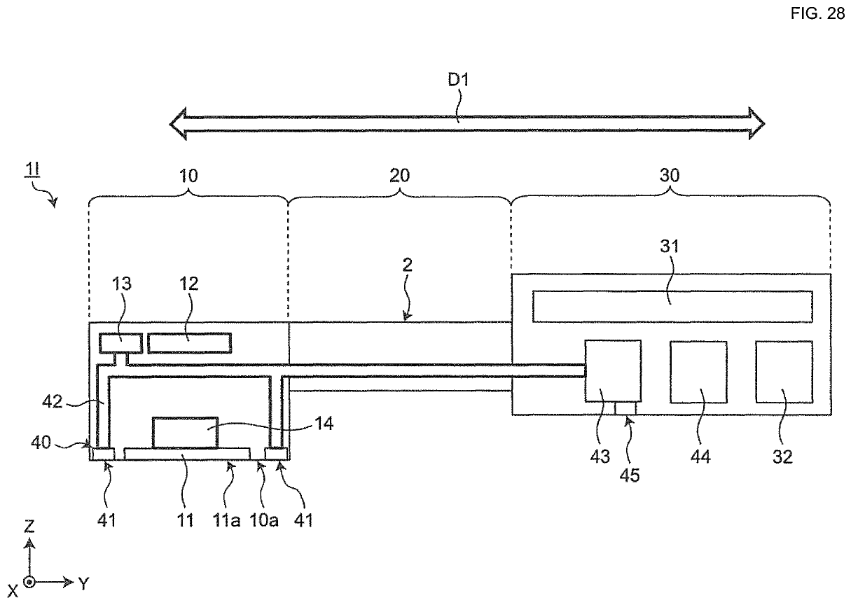

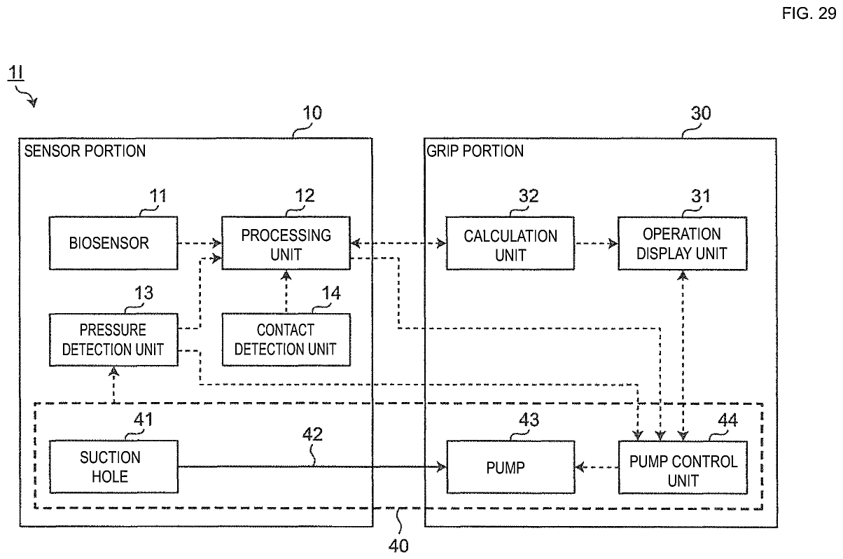

An example of a measurement device of Embodiment 9 will be described with reference to FIG. 28 and FIG. 29. FIG. 28 is a schematic diagram of an internal configuration of an example of a measurement device 1I of Embodiment 9 according to the present invention. FIG. 29 is a block diagram of a schematic configuration of an example of the measurement device 1I of Embodiment 9 according to the present invention.

Embodiment 9 is different from Embodiment 8 in that a contact detection unit 14 is included.

As illustrated in FIG. 28 and FIG. 29, the measurement device 1I includes the contact detection unit 14. The contact detection unit 14 detects contact information indicating a degree of contact between the biosensor 11 and a living body.

In Embodiment 9, the contact detection unit 14 is a load sensor. The load sensor detects the load applied to the biosensor 11. That is, the contact detection unit 14 acquires the load applied to the biosensor 11 as the contact information.

The contact detection unit 14 is disposed in the sensor portion 10.

The contact information detected by the contact detection unit 14 is transmitted to the processing unit 12.

For example, the contact detection unit 14 is controlled by a control unit included in the measurement device 1I.

The processing unit 12 receives the contact information from the contact detection unit 14 and determines whether or not the biosensor 11 and a living body are in contact with each other on the basis of the contact information. For example, when the load detected by the contact detection unit 14 exceeds a predetermined threshold value, the processing unit 12 determines that the biosensor 11 and the living body are in contact with each other.

Upon determining that the biosensor 11 is in contact with the living body, the processing unit 12 generates and outputs trigger information for starting suction. The trigger information for starting suction generated by the processing unit 12 is transmitted to the pump control unit 44. The pump control unit 44 receives the trigger information for starting suction from the processing unit 12, and starts sucking on the basis of the trigger information for starting suction.

As described above, the suction portion 40 starts sucking on the basis of the contact information detected by the contact detection unit 14.

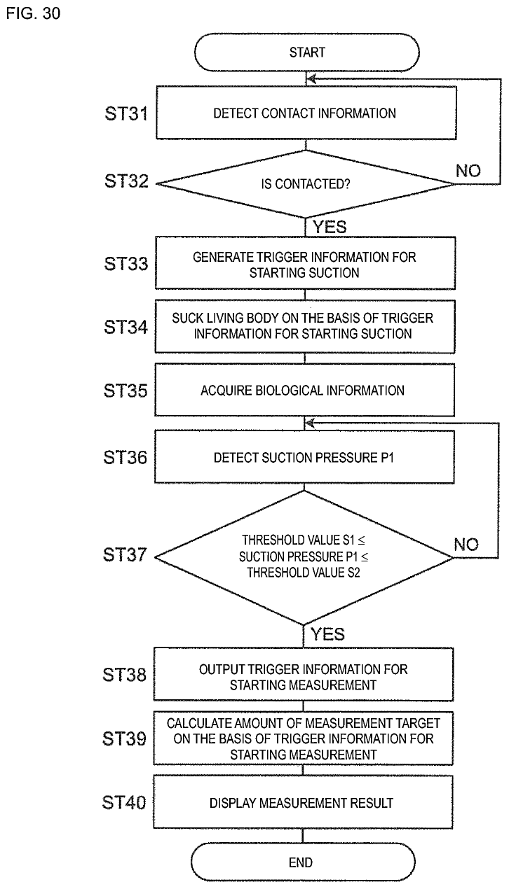

FIG. 30 is a flowchart of an example of an operation of the measurement device 1I of Embodiment 9 according to the present invention. Since steps ST35 to ST40 in FIG. 30 are the same as steps ST22 to ST27 in FIG. 27 of Embodiment 8, detailed description thereof will be omitted.

As illustrated in FIG. 30, in step ST31, contact information is detected by the contact detection unit 14. In Embodiment 9, the contact detection unit 14 is a load sensor, and the contact information is a load applied to the biosensor 11 with the biosensor 11 coming into contact with a living body. The contact information detected by the contact detection unit 14 is transmitted to the processing unit 12.

In step ST32, the processing unit 12 determines whether or not the biosensor 11 is in contact with a living body. The processing unit 12 receives the contact information from the contact detection unit 14 and determines whether or not the biosensor 11 and a living body are in contact with each other on the basis of the contact information. For example, when the load detected by the contact detection unit 14 exceeds a predetermined threshold value, the processing unit 12 determines that the biosensor 11 and a living body are in contact with each other.

The load is used for the determination in step ST32, but the present invention is not limited thereto. The determination in step ST32 may use a mean value, a median value, a minimum value, or a maximum value of the load in exemplary aspects.

When the processing unit 12 determines that the biosensor 11 and a living body are in contact with each other, the flow proceeds to step ST33. When the processing unit 12 determines that the biosensor 11 is not in contact with the living body, the flow returns to step ST31.

In step ST33, the processing unit 12 generates trigger information for starting suction. The processing unit 12 transmits trigger information for starting suction to the pump control unit 44 of the suction portion 40.

In step ST34, the suction portion 40 sucks the living body on the basis of the trigger information for starting suction. In the suction portion 40, the pump control unit 44 receives trigger information for starting suction from the processing unit 12, and controls the pump 43 on the basis of the trigger information.

In step ST35, the biosensor 11 acquires biological information. The biological information acquired by the biosensor 11 is transmitted to the processing unit 12. The processing unit 12 converts the biological information and transmits the converted information to the calculation unit 32.

In step ST36, the pressure detection unit 13 detects the suction pressure P1. Information on the suction pressure P1 detected by the pressure detection unit 13 is transmitted to the processing unit 12.

In step ST37, the processing unit 12 determines whether or not the suction pressure P1 is within a predetermined range.

When the processing unit 12 determines in step ST37 that the suction pressure P1 is in the range of the first threshold value S1 or more and the second threshold value or less, the flow proceeds to step ST38. When the processing unit 12 determines that the suction pressure P1 is out of the range of the first threshold value S1 or more and the second threshold value or less, the flow returns to step ST36.

In step ST38, the processing unit 12 generates trigger information for starting measurement. The processing unit 12 transmits the trigger information for starting measurement to the calculation unit 32.

In step ST39, the calculation unit 32 calculates the amount of the measurement target on the basis of the trigger information.

In step ST40, the measurement result is displayed with the operation display unit 31. The operation display unit 31 receives the information on the amount of the measurement target from the calculation unit 32, and displays the information.

As described above, by performing steps ST31 to ST40, the measurement device 1I may start sucking a living body after detecting contact between the biosensor 11 and the living body, and calculate the amount of the measurement target.

### Technical Effects

With the use of the measurement device 1I according to Embodiment 9, the following effects may be exhibited.

The measurement device 1I includes the contact detection unit 14 that detects contact information between the biosensor 11 and a living body. The suction portion 40 starts sucking on the basis of the contact information detected by the contact detection unit 14. With the configuration above, sucking by the suction portion 40 may be started after the biosensor 11 and the living body are brought into contact with each other. This configuration improves the usability of the measurement device 1I. Further, since the measurement may be started after the biosensor 11 and the living body are brought into contact with each other, the variation in the measurement may be suppressed and the measurement accuracy may be increased.

Note that, in Embodiment 9, an example has been described in which the contact information detected by the contact detection unit 14 is used to generate the trigger information for starting suction, but the present invention is not limited thereto. For example, the contact information detected by the contact detection unit 14 may be used to generate the trigger information for starting measurement. That is, the processing unit 12 may generate the trigger information for starting measurement on the basis of the information on the suction pressure P1 detected by the pressure detection unit 13 and the contact information detected by the contact detection unit 14.

In Embodiment 9, an example has been described in which the contact detection unit 14 is a load sensor, but the present invention is not limited thereto. It is sufficient that the contact detection unit 14 is a sensor configured to detect the contact information indicating a degree of contact between the biosensor 11 and a living body. For example, the contact detection unit 14 may be such as an optical sensor, a distance measurement sensor, or a temperature sensor.

In Embodiment 9, an example has been described in which the contact detection unit 14 is disposed in the sensor portion 10, but the present invention is not limited thereto. For example, the contact detection unit 14 may be disposed in the probe portion 20 in another exemplary aspect.

The contact detection unit 14 may be disposed on a main circuit substrate including the calculation unit 32. In the case above, the contact information detected by the contact detection unit 14 may directly be transmitted to the calculation unit 32. The calculation unit 32 may execute the process of the processing unit 12 described in Embodiment 9. Alternatively, both the processing unit 12 and the calculation unit 32 may execute the process of the processing unit 12 described in Embodiment 9.

In Embodiment 9, an example has been described in which the processing unit 12 determines whether or not the biosensor 11 and a living body are in contact with each other on the basis of the contact information, but the present invention is not limited thereto. For example, the pump control unit 44 may determine whether or not the biosensor 11 and a living body are in contact with each other on the basis of the contact information. In the case above, the contact information detected by the contact detection unit 14 may be transmitted to the pump control unit 44. The trigger information for starting suction may be generated by the pump control unit 44.

In Embodiment 9, an example has been described in which the measurement device 1I includes the pressure detection unit 13, but the present invention is not limited thereto. The measurement device 1I is not required to include the pressure detection unit 13.

In Embodiment 9, an example has been described in which the measurement device 1I includes the calculation unit 32, but the present invention is not limited thereto. For example, the calculation unit 32 may be included in a device different from the measurement device 1I.

### Embodiment 10

A measurement device according to Embodiment 10 of an exemplary aspect will be described. Note that, in Embodiment 10, different points from Embodiment 9 will mainly be described. In Embodiment 10, components identical or equivalent to those in Embodiment 9 are denoted by the same reference signs. Further, in Embodiment 10, a description overlapping with Embodiment 9 will be omitted.

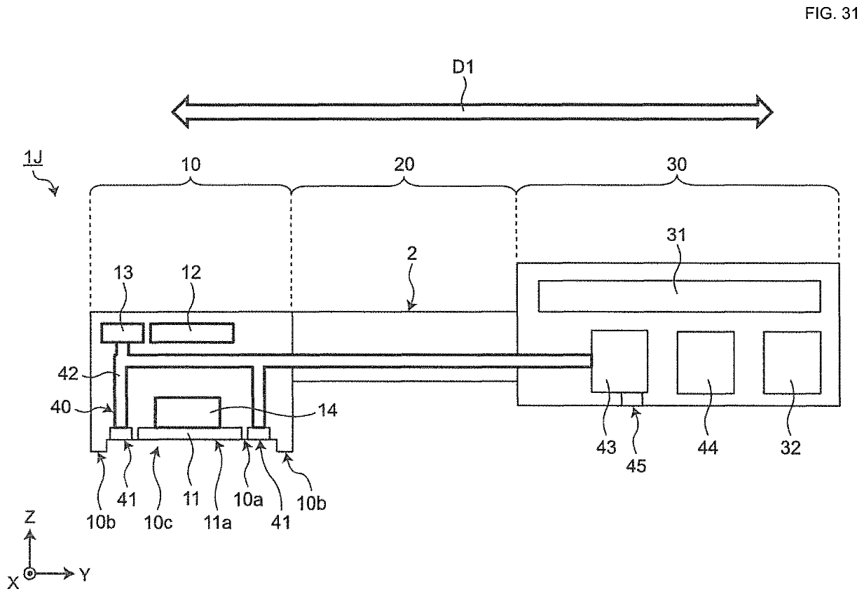

An example of a measurement device of Embodiment 10 will be described with reference to FIG. 31 and FIG. 32. FIG. 31 is a schematic diagram of an internal configuration of an example of a measurement device 1J of Embodiment 10 according to the present invention. FIG. 32 is a schematic enlarged diagram of part of the measurement device 1J of Embodiment 10 according to the present invention.

Embodiment 10 is different from Embodiment 9 in that a step portion 10b is provided on the contact surface 10a.

In particular, as illustrated in FIG. 31 and FIG. 32, the measurement device 1J has the step portion 10b (or simply referred to as a “step”) on the contact surface 10a. The step portion 10b protrudes from the contact surface 10a toward outside the measurement device 1J, and is provided on the periphery of the biosensor 11 and the multiple suction holes 41. Outside the measurement device 1J from the contact surface 10a refers to a direction away from the contact surface 10a.

The step portion 10b is formed in a frame shape. When viewed from the height direction (i.e., in the Z direction) of the measurement device 1J, the step portion 10b is provided along the outer periphery of the contact surface 10a. A recessed portion 10c is formed inside the step portion 10b. The biosensor 11 is disposed on the recessed surface of the recessed portion 10c. Further, the recessed surface is provided with the multiple suction holes 41.

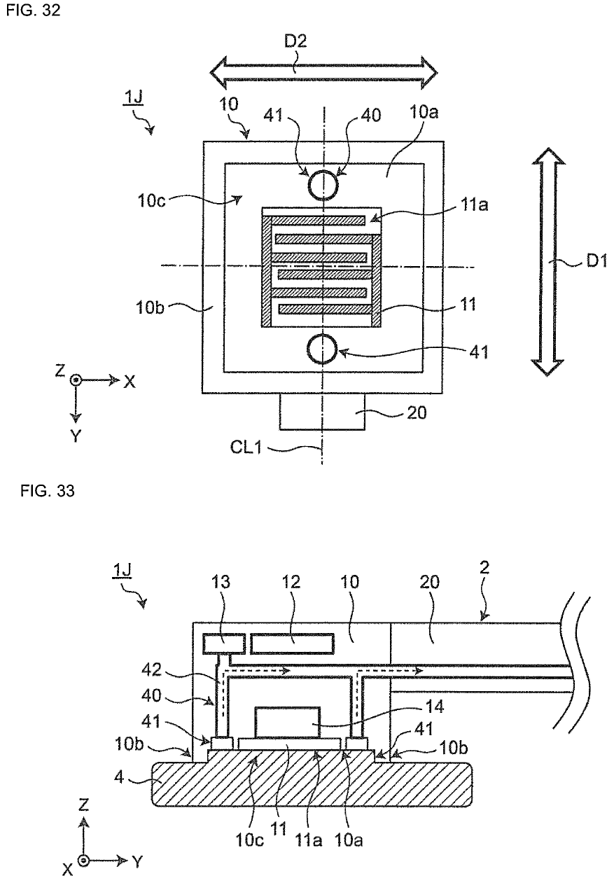

FIG. 33 is a schematic diagram of an example of a state in which the measurement device 1J of Embodiment 10 according to the present invention is used. FIG. 33 illustrates a state in which the suction portion 40 sucks the living body 4 from the multiple suction holes 41. As illustrated in FIG. 33, when the sensor portion 10 is brought into contact with the living body 4, the opening on the recessed portion 10c is covered by the living body 4 to form a closed space. In the state above, when the suction portion 40 sucks the living body 4 from the multiple suction holes 41, the living body 4 deforms and enters into the recessed portion 10c of the step portion 10b. With this, the detection surface 11a of the biosensor 11 and the living body 4 may more easily be brought into contact with each other.

The deformation of the living body 4 is determined by the shape and size of the step portion 10b. In other words, the deformation of the living body 4 is determined by the shape and size of the recessed portion 10c. Thus, by appropriately designing the shape and size of the step portion 10b, damage to the living body 4 may be suppressed.

For example, the height of the step portion 10b is 0.050 mm or more and 2.0 mm or less. With the configuration above, the detection surface 11a of the biosensor 11 and the living body 4 may more easily be brought into contact with each other while suppressing damage to the living body 4.

### Technical Effects

With the use of the measurement device 1J according to Embodiment 10, the following effects may be exhibited.

The measurement device 1J includes the step portion 10b protruding from the contact surface 10a toward outside the measurement device 1J and being provided on the periphery of the biosensor 11 and the multiple suction holes 41. With the configuration above, the detection surface 11a of the biosensor 11 and the living body 4 may more easily be brought into contact with each other, and the measurement accuracy may be increased.

With the use of the measurement device 1J, when the measurement device 1J comes into contact with the living body 4, a closed space may be formed inside the step portion 10b, and the living body 4 may be sucked from the multiple suction holes 41 in the closed space. With this, the suction force of the multiple suction holes 41 may be made uniform, and the detection surface 11a of the biosensor 11 and the living body 4 may more easily be brought into contact with each other. Further, the contact state may more easily be maintained.

With the use of the measurement device 1J, by appropriately designing the shape and size of the step portion 10b, the measurement accuracy may be increased while suppressing damage to the living body 4. For example, the deformation amount of the living body 4 to be sucked may be determined by the height of the step portion 10b. Since the damage given to the living body 4 is determined by the deformation amount of the living body 4, when the height of the step portion 10b is lowered, restrain of a suction error may be achieved while suppressing damage to the living body 4. With this, a highly accurate measurement can be performed on various patients with a low load.

Note that, in Embodiment 10, an example has been described in which the step portion 10b is provided along the outer periphery of the contact surface 10a, but the present invention is not limited thereto. It is sufficient that the step portion 10b is provided on the contact surface 10a to surround the periphery of the biosensor 11 and the multiple suction holes 41.

In Embodiment 10, an example has been described in which the step portion 10b is formed in a frame shape, but the present invention is not limited thereto. For example, the step portion 10b may be formed in an annular shape. Alternatively, the frame-shaped step portion 10b may be formed by multiple parts.

In Embodiment 10, an example has been described in which the multiple suction holes 41 are provided in the contact surface 10a, but the present invention is not limited thereto. It is sufficient that one or multiple suction holes 41 are provided in the contact surface 10a.

### Embodiment 11

A measurement device according to Embodiment 11 of an exemplary aspect will be described. Note that, in Embodiment 11, different points from Embodiment 9 will mainly be described. In Embodiment 11, components identical or equivalent to those in Embodiment 9 are denoted by the same reference signs. Further, in Embodiment 11, a description overlapping with Embodiment 9 will be omitted.

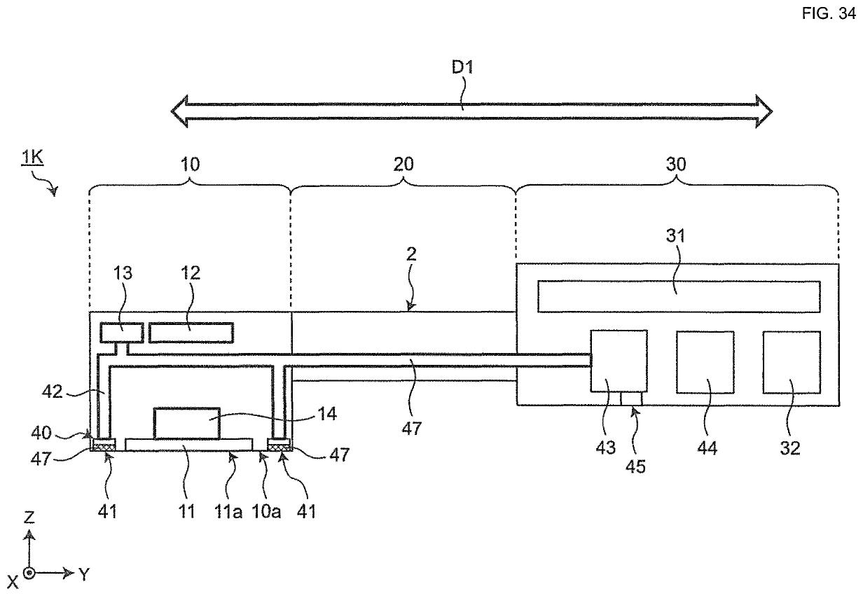

An example of a measurement device of Embodiment 11 will be described with reference to FIG. 34. FIG. 34 is a schematic diagram of an internal configuration of an example of a measurement device 1K of Embodiment 11 according to the present invention.

Embodiment 11 is different from Embodiment 9 in that a filter 47 is included.

As illustrated in FIG. 34, the measurement device 1K includes the multiple filters 47 that are disposed in the multiple suction holes 41.

The filter 47 isolates liquid and gas from each other. For example, the filter 47 is a hydrophobic air-permeable membrane. By disposing the filter 47 in the suction hole 41, the inflow of liquid into the measurement device 1K is suppressed.

### Technical Effects

With the use of the measurement device 1K according to Embodiment 11, the following effects may be exhibited.

The measurement device 1K includes the multiple filters 47 disposed in the multiple suction holes 41 and isolating liquid and gas from each other. With the configuration above, the inflow of liquid into the measurement device 1K may be suppressed, and a failure and/or contamination of the measurement device 1K due to liquid may be suppressed.

Further, with the use of the measurement device 1K, measurement may be performed with the cover film 3 being not attached.

The measurement device 1K may have a configuration in which the sensor portion 10 is replaceable. After completion of a measurement, the sensor portion 10 may be detached from the probe portion 20 and a new sensor portion 10 may be attached to replace the original sensor portion 10.

Note that, in Embodiment 11, an example has been described in which the measurement device 1K includes the multiple filters 47, but the present invention is not limited thereto. It is sufficient that the measurement device 1K includes one or more filters 47. For example, in a case that one suction hole 41 is provided in the contact surface 10a, it is sufficient that the measurement device 1K includes one filter 47.

In Embodiment 11, an example has been described in which the filter 47 is disposed in the suction hole 41, but the present invention is not limited thereto. For example, the filter 47 may be disposed in the suction path 42.

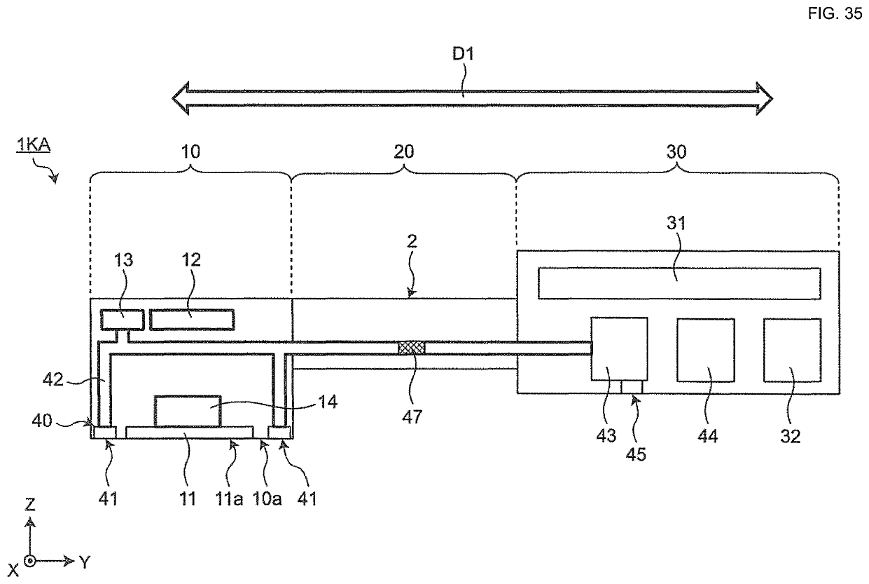

FIG. 35 is a schematic diagram of an internal configuration of a measurement device 1KA of a modification of Embodiment 11 according to an exemplary aspect. As illustrated in FIG. 35, the measurement device 1KA includes the filter 47 disposed inside the suction path 42. The filter 47 is disposed in the suction path 42 positioned in the probe portion 20. Even in the configuration above, a failure and/or contamination of the measurement device 1K due to liquid may be suppressed.

### Embodiment 12

A measurement device according to Embodiment 12 of an exemplary aspect will be described. Note that, in Embodiment 12, different points from Embodiment 1 will mainly be described. In Embodiment 12, components identical or equivalent to those in Embodiment 1 are denoted by the same reference signs. Further, in Embodiment 12, a description overlapping with Embodiment 1 will be omitted.

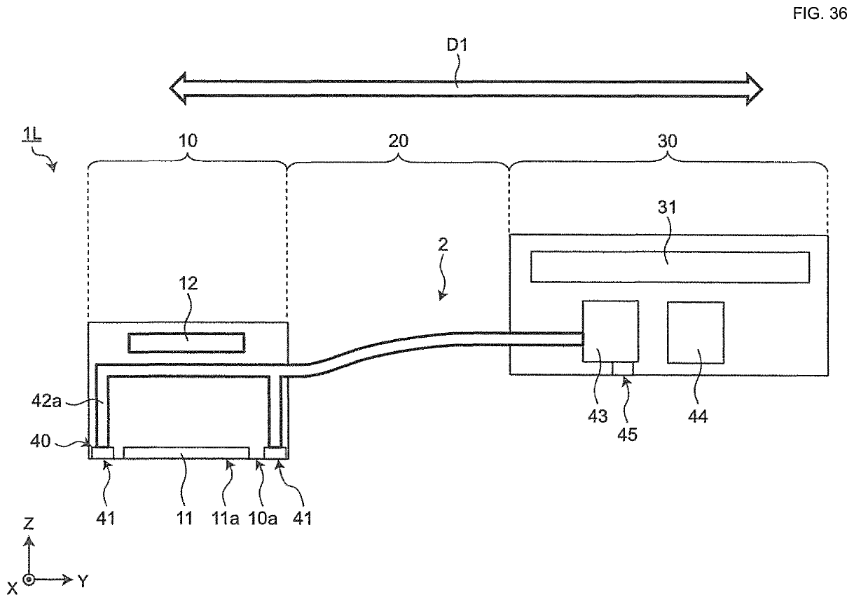

An example of a measurement device of Embodiment 12 will be described with reference to FIG. 36. FIG. 36 is a schematic diagram of an internal configuration of an example of a measurement device 1L of Embodiment 12 according to the present invention.

Embodiment 12 is different from Embodiment 1 in that the housing 2 includes the sensor portion 10, a tube 20a, and a main body portion 30a.

As illustrated in FIG. 36, the housing 2 of the measurement device 1L includes the sensor portion 10, the tube 20a, and the main body portion 30a. Since the sensor portion 10 is the same as that in Embodiment 1, a description thereof will be omitted.

The tube 20a connects the sensor portion 10 and the main body portion 30a. The tube 20a forms part of the suction path 42 of Embodiment 1. The tube 20a is flexibly deformable.

The main body portion 30a includes the operation display unit 31, the pump 43, and the pump control unit 44 of Embodiment 1. Further, part of the suction path 42 is formed inside the main body portion 30a.

### Technical Effects

With the use of the measurement device 1L according to Embodiment 12, the following effects may be exhibited.

In the measurement device 1L, the housing 2 includes the sensor portion 10, the tube 20a, and the main body portion 30a. The biosensor 11 is disposed in the sensor portion 10. The tube 20a forms part of the suction path 42, and connects the sensor portion 10 and the main body portion 30a. The pump 43 and the pump control unit 44 are disposed in the main body portion 30a. With the configuration above, since the contact surface 10a may easily be brought into contact with a living body, the usability of the measurement device 1L increases.

Further, in a case in which a measurement is performed in the oral cavity, the measurement may be performed with one's hands free.

### Embodiment 13

A measurement system according to Embodiment 13 of an exemplary aspect will be described. Note that, in Embodiment 13, different points from Embodiment 1 will mainly be described. In Embodiment 13, components identical or equivalent to those in Embodiment 1 are denoted by the same reference signs. Further, in Embodiment 13, a description overlapping with Embodiment 1 will be omitted.

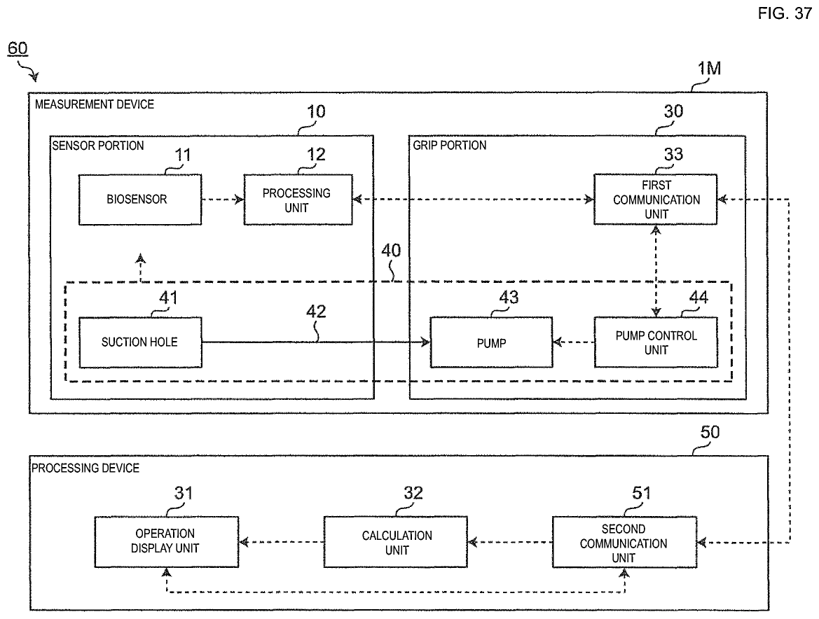

An example of a measurement system of Embodiment 13 will be described with reference to FIG. 37. FIG. 37 is a block diagram of a schematic configuration of an example of a measurement system 60 of Embodiment 13 according to the exemplary aspect.

Embodiment 13 is different from Embodiment 1 in that information acquired by a measurement device 1M is transmitted to a processing device 50, and the amount of the measurement target is calculated by the processing device 50.

As illustrated in FIG. 37, the measurement system 60 includes the measurement device 1M and the processing device 50. In Embodiment 13, an example will be described in which the measurement system 60 is an intraoral measurement system.

<Measurement Device>

The measurement device 1M includes the biosensor 11, the processing unit 12, and a first communication unit 33. In Embodiment 13, since the biosensor 11 and the processing unit 12 are the same as those in Embodiment 1, detailed description thereof will be omitted.

The first communication unit 33 communicates with the processing device 50. The first communication unit 33 transmits the biological information to the processing device 50. In Embodiment 13, the processing unit 12 converts the biological information acquired by the biosensor 11. Thus, the first communication unit 33 transmits the information converted by the processing unit 12 to the processing device 50.

The first communication unit 33 includes a circuit that communicates with the processing device 50 in conformity with a predetermined communication standard. The predetermined communication standard includes LAN, Wi-Fi®, Bluetooth®, USB, HDMI®, controller area network (CAN), serial peripheral interface (SPI), universal asynchronous receiver/transmitter (UART), and inter-integrated circuit (I2C), for example.

The measurement device 1M includes a first control unit that integrally controls components constituting the measurement device 1M. The first control unit includes a memory storing a program and a processing circuit corresponding to a processor such as a central processing unit (CPU), for example. For example, in the first control unit, the processor executes a program stored in the memory. In Embodiment 13, the first control unit controls the biosensor 11, the processing unit 12, and the first communication unit 33.

In Embodiment 13, the biosensor 11 is an electrostatic capacity sensor, and acquires electrostatic capacity as biological information. The processing unit 12 converts the electrostatic capacity into frequency information with the frequency conversion circuit. The first communication unit 33 transmits the frequency information converted by the processing unit 12 to the processing device 50.

<Processing Device>

In operation, the processing device 50 receives the information from the measurement device 1M, and calculates the amount of the measurement target on the basis of the received information. In Embodiment 13, the processing device 50 calculates the moisture amount on the basis of the frequency information received from the measurement device 1M.

The processing device 50 is a computer. For example, the processing device 50 may be a portable terminal, such as a smartphone or a tablet terminal. Alternatively, the processing device 50 may be a server connected to a network.

The processing device 50 includes a second communication unit 51, the operation display unit 31, and the calculation unit 32. In Embodiment 13, since the operation display unit 31 and the calculation unit 32 are the same as those in Embodiment 1 and Embodiment 7, detailed description thereof will be omitted.

The second communication unit 51 communicates with the measurement device 1M. Specifically, the second communication unit 51 receives the biological information from the first communication unit 33 of the measurement device 1M.

The second communication unit 51 includes a circuit that communicates with the measurement device 1M in conformity with a predetermined communication standard. The predetermined communication standard includes LAN, Wi-Fi®, Bluetooth®, USB, HDMI®, controller area network (CAN), serial peripheral interface (SPI), universal asynchronous receiver/transmitter (UART), and inter-integrated circuit (I2C), for example.

The processing device 50 receives biological information from the measurement device 1M via the second communication unit 51. In Embodiment 13, the processing device 50 receives the frequency information from the measurement device 1M via the second communication unit 51.

In the processing device 50, the calculation unit 32 calculates the amount of the measurement target on the basis of the biological information received from the measurement device 1M. In Embodiment 13, the calculation unit 32 calculates the moisture amount on the basis of the frequency information. The information on the calculated moisture amount is transmitted to the operation display unit 31. The operation display unit 31 displays the information on the calculated moisture amount.

The processing device 50 includes a second control unit to integrally control the components constituting the processing device 50. The second control unit includes a memory storing a program and a processing circuit corresponding to a processor such as a central processing unit (CPU), for example. For example, in the second control unit, the processor executes a program stored in the memory. In Embodiment 13, the second control unit controls the second communication unit 51, the operation display unit 31, and the calculation unit 32.

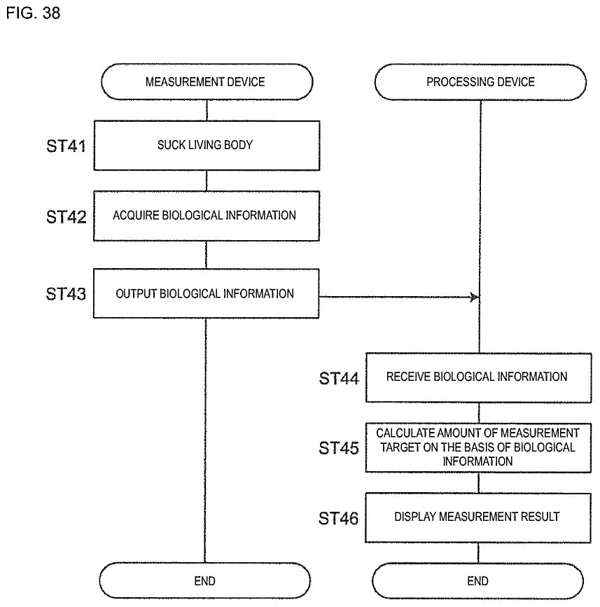

FIG. 38 is a flowchart of an example of an operation of the measurement system 60 of Embodiment 13 according to an exemplary aspect. Since steps ST41 to ST43 in FIG. 38 are the same as steps ST1 to ST3 in FIG. 5 of Embodiment 1, detailed description thereof will be omitted.

As illustrated in FIG. 38, in step ST41, the suction portion 40 sucks a living body.

In step ST42, the biosensor 11 acquires biological information. The biological information acquired by the biosensor 11 is transmitted to the processing unit 12. The processing unit 12 converts the biological information and transmits the converted information to the first communication unit 33.

In Embodiment 13, the biosensor 11 is an electrostatic capacity sensor. The biosensor 11 acquires information on electrostatic capacity as biological information. Further, the biosensor 11 transmits the information on the electrostatic capacity to the processing unit 12. The processing unit 12 receives the information on the electrostatic capacity from the biosensor 11, and converts the electrostatic capacity into frequency with the frequency conversion circuit. Further, the processing unit 12 continues converting while receiving the information on the electrostatic capacity from the biosensor 11, and continues storing the converted information in the storage unit included in the measurement device 1M. The processing unit 12 transmits the information stored in the storage unit to the first communication unit 33.

In step ST43, the biological information is outputted by the first communication unit 33.

In Embodiment 13, the first communication unit 33 transmits the information converted by the processing unit 12 to the processing device 50.

In step ST44, the biological information is received by the second communication unit 51 of the processing device 50. Specifically, the processing device 50 receives the biological information from the measurement device 1M via the second communication unit 51. The biological information received by the second communication unit 51 is transmitted to the calculation unit 32.

In step ST45, the calculation unit 32 calculates the amount of the measurement target on the basis of the biological information. Information on the amount of the measurement target calculated by the calculation unit 32 is transmitted to the operation display unit 31.

In Embodiment 13, the calculation unit 32 calculates the amount of the measurement target on the basis of the information converted by the processing unit 12. Specifically, the calculation unit 32 calculates the moisture amount on the basis of the frequency.

In step ST46, the operation display unit 31 displays the measurement result. The operation display unit 31 receives the information on the amount of the measurement target from the calculation unit 32, and displays the information.

As described above, by performing steps ST41 to ST46, the measurement system 60 may calculate the amount of the measurement target.

[Technical Effects]

With the use of the measurement system 60 according to Embodiment 13, the following effects may be exhibited.

The measurement system 60 includes the measurement device 1M having the contact surface 10a that comes into contact with a measurement portion of a living body and the processing device 50 that communicates with the measurement device 1M. The measurement device 1M includes the biosensor 11, the suction portion 40, and the first communication unit 33. The biosensor 11 is disposed on the contact surface 10a and has the detection surface 11a that acquires biological information. The suction portion 40 sucks a living body from one or multiple suction holes 41 provided on the periphery of the detection surface 11a of the biosensor 11 on the contact surface 10a. The first communication unit 33 transmits the biological information to the processing device 50. The processing device 50 includes the second communication unit 51 and the calculation unit 32. The second communication unit 51 receives the biological information from the first communication unit 33 of the measurement device 1M. The calculation unit 32 calculates the amount of the measurement target on the basis of the biological information.

With the configuration above, the measurement accuracy may be increased as in Embodiment 1. With the use of the measurement system 60, by sucking a living body with the suction portion 40, the living body is easily brought into contact with the detection surface 11a of the biosensor 11. Further, with the suction force of the suction portion 40, the contact between the detection surface 11a of the biosensor 11 and a living body may easily be maintained.

Note that, in Embodiment 13, an example has been described in which the processing device 50 includes the operation display unit 31, but the present invention is not limited thereto. In the processing device 50, the operation display unit 31 is not an essential component. For example, the operation display unit 31 may be provided in the measurement device 1M. Alternatively, the operation display unit 31 may be provided in a different external apparatus. The input information inputted to the operation display unit 31 may be transmitted to the measurement device 1M via the second communication unit 51.

In Embodiment 13, an example has been described in which the measurement target of the measurement system 60 is moisture, but the measurement target is not limited thereto. It is sufficient that the measurement system 60 is configured to measure the amount of a measurement target of a living body.

In Embodiment 13, an example has been described in which the measurement system 60 includes the measurement device 1M, but the present invention is not limited thereto. The measurement system 60 may include the measurement devices of Embodiment 2 to Embodiment 6.

Although the present invention has been fully described in connection with the exemplary embodiments with reference to the accompanying drawings, various changes and modifications are apparent to those skilled in the art. It is to be understood that such variations and modifications are intended to be included within the scope of the present invention as long as they do not depart from the scope.

The exemplary measurement device and the measurement system of the present invention may be applied to such as a moisture amount measurement device to measure moisture amount in the oral cavity, for example.

### REFERENCE SIGNS LIST

1A, 1AA, 1AB, 1AC, 1AD, 1B, 1BA, 1C, 1CA, 1D, 1E, 1F, 1FA, 1G, 1GA, 1H, 1I, 1J, 1K, 1KA, 1L, 1M MEASUREMENT DEVICE
2 HOUSING
3 COVER FILM
3a MEMBRANE PORTION
4 LIVING BODY
5 EXTERNAL DEVICE
10 SENSOR PORTION
10a CONTACT SURFACE
10b STEP PORTION
10c RECESSED PORTION
11 BIOSENSOR
11a DETECTION SURFACE
12 PROCESSING UNIT
13 PRESSURE DETECTION UNIT
14 CONTACT DETECTION UNIT
20 PROBE PORTION
20a TUBE
30 GRIP PORTION
30a MAIN BODY PORTION
31 OPERATION DISPLAY UNIT
32 CALCULATION UNIT
33 FIRST COMMUNICATION UNIT
40 SUCTION PORTION
41, 41a, 41b, 41c, 41d, 41e, 41f, 41g, 41h, 41i SUCTION HOLE
42 SUCTION PATH
43 PUMP
44 PUMP CONTROL UNIT
45 EXHAUST HOLE
46, 46a SENSOR SUCTION HOLE
47 FILTER
50 PROCESSING DEVICE
51 SECOND COMMUNICATION UNIT
60 MEASUREMENT SYSTEM

1. A measurement device comprising:
a contact surface configured to contact a measurement portion of a living body;
a biosensor disposed on the contact surface and having a detection surface configured to acquire biological information from the measurement portion; and
a suction portion configured to suck the living body from at least one suction hole provided in the contact surface on a periphery of the detection surface of the biosensor.

2. The measurement device according to claim 1, further comprising:
a housing having a longer direction and a shorter direction that is orthogonal to the longer direction,
wherein the housing includes:
a sensor disposed on one end side in the longer direction, and
a grip disposed on another end side in the longer direction.

3. The measurement device according to claim 2, wherein the biosensor is disposed in the sensor, and the at least one suction hole comprises multiple suction holes that sandwich the biosensor in the longer direction.

4. The measurement device according to claim 2, wherein the biosensor is disposed in the sensor, and the at least one suction hole comprises multiple suction holes that sandwich the biosensor in the shorter direction that is orthogonal to the longer direction.

5. The measurement device according to claim 1, wherein the suction portion is configured to suck the living body from at least one sensor suction hole disposed in the detection surface of the biosensor.

6. The measurement device according to claim 1, wherein the detection surface of the biosensor has a polygonal shape, and the at least one suction hole comprises multiple suction holes that are disposed in corner portions of the detection surface.

7. The measurement device according to claim 1, wherein the at least one suction hole comprises multiple suction holes that are symmetrically disposed with respect to the biosensor.

8. The measurement device according to claim 1, wherein the suction portion includes:
a pump configured to suck gas,
a suction path connecting the at least one suction hole to the pump, and
at least one filter disposed in at least one of the at least one suction hole and the suction path,
wherein the at least one filter is configured to isolate liquid and gas from each other.

9. The measurement device according to claim 8, wherein the at least one filter is a hydrophobic air-permeable membrane.

10. The measurement device according to claim 1, further comprising a step portion that protrudes from the contact surface towards an outside the measurement device and is disposed on a periphery of the biosensor and the at least one suction hole.

11. The measurement device according to claim 1, further comprising a calculation unit configured to calculate an amount of a measurement target based on the biological information acquired by the biosensor.

12. The measurement device according to claim 11, wherein the amount of the measurement target is a moisture amount.

13. The measurement device according to claim 1, further comprising:
a pressure detection unit configured to detect a suction pressure with which the suction portion sucks the living body; and
a processing unit configured to output trigger information for starting a measurement based on the suction pressure detected by the pressure detection unit.

14. The measurement device according to claim 13, wherein the processing unit is configured to output the trigger information for starting the measurement when the suction pressure is 10 kPa or more and 40 kPa or less.

15. The measurement device according to claim 13,
wherein the biosensor is an electrostatic capacity sensor configured to detect electrostatic capacity, and
the processing unit is configured to convert the electrostatic capacity detected by the electrostatic capacity sensor into a frequency.

16. The measurement device according to claim 1, further comprising:
a contact detection unit configured to detect contact information between the biosensor and the living body,
wherein the suction portion is configured to start sucking the living body based on contact information detected by the contact detection unit.

17. The measurement device according to claim 1, further comprising:
a cover film that covers the biosensor and the at least one suction hole,
wherein the cover film has a membrane that isolates liquid from gas.

18. The measurement device according to claim 1, wherein the measurement portion of the living body is a measurement portion in an oral cavity.

19. The measurement device according to claim 1,
wherein the biosensor comprises a comb-shaped electrode having a plurality of arms extending in a first direction, and
wherein the at least one suction hole comprises at least a pair of suction holes that sandwich the comb-shaped electrode in a second direction orthogonal to the first direction.

20. A measurement system, comprising:
a measurement device having a contact surface configured to contact a measurement portion of a living body; and
a processing device configured to communicate with the measurement device, wherein the measurement device includes:
a biosensor disposed on the contact surface and having a detection surface configured to acquire biological information from the measurement portion,
a suction portion configured to suck the living body from at least one suction hole disposed in the contact surface on a periphery of the detection surface of the biosensor, and
a first communication unit configured to transmit the biological information to the processing device, and
wherein the processing device includes:
a second communication unit configured to receive the biological information from the first communication unit of the measurement device, and
a calculation unit configured to calculate an amount of a measurement target based on the received biological information.
